(19) United States
(12) Patent Application Publication
(10) Pub. No.: US 2025/0161248 A1
(43) Pub. Date: May 22, 2025

(54) Treating Chronic Myelomonocytic Leukemia with CXCL8 Receptor Inhibitors

(71) Applicants: Institut Gustave Roussy, Villejuif (FR); Universite Paris-Saclay, Gif-sur-Yvette (FR); Institut National de la Sante et de la Recherche Medicale (INSERM), Paris (FR)

(72) Inventors: Dorothee SELIMOGLU-BUET, Villejuif (FR); Eric SOLARY, Villejuif (FR); Christophe WILLEKENS, Villejuif (FR)

(73) Assignees: Institut Gustave Roussy, Villejuif (FR); Universite Paris-Saclay, Gif-sur-Yvette (FR); Institut National de la Sante et de la Recherche Medicale (INSERM), Paris (FR)

(21) Appl. No.: 18/382,280

(22) Filed: Oct. 20, 2023

(51) Int. Cl. A61K 31/18; A61K 45/06; A61P 35/02; G01N 33/68

(52) U.S. Cl. CPC A61K 31/18; A61K 45/06; A61P 35/02; G01N 33/6869; G01N 2333/5421; G01N 2800/52

(57) ABSTRACT

A method for treating chronic myelomonocytic leukemia (CMML), or for maintaining or enhancing the proliferation of wildtype hematopoietic stem cells in patients suffering from CMML, by administering to a patient in need thereof a pharmaceutical composition containing an effective amount of an inhibitor of the CXCL8 receptor.

### BACKGROUND

Chronic myelomonocytic leukemia (CMML) is a myeloid malignancy defined by overlapping features of both myeloproliferative and myelodysplastic neoplasms (1). CMML diagnosis is based on sustained absolute and relative peripheral blood monocytosis with abnormal partitioning of peripheral blood monocyte subsets, one of more clonal cytogenetic or molecular abnormalities, and/or dysplasia in at least one lineage (2). The disease predominantly affects elderly patients, and mutational signatures of leukemic cells suggest that ageing is the main cause of the disease (3). Multiple studies have substantiated the clinical and molecular distinction of dysplastic (MD-CMML) and proliferative (MP-CMML) CMML on the basis of cutoff white blood cell (WBC) count of 13×109/L (4). Blast cell count in the bone marrow also separates CMML in subgroups with distinct outcomes (5). CMML is difficult to treat (1). Allogeneic stem cell transplantation, which is the only potentially curative treatment, is commonly precluded by age and comorbidities, and only partially abrogates the risk of relapse (6). In patients with MD-CMML, hypomethylating agents can restore balanced hematopoiesis but do not reduce the variant allele frequency among circulating myeloid cells (3) and do not prevent progression to acute myeloid leukemia (1). In patients with MP-CMML, hypomethylating agents do not demonstrate a survival benefit compared to cytoreductive therapy with hydroxyurea (7).

Therefore, there is an urgent need for additional therapeutic approaches for this disease.

CMML is a clonal disorder driven by the linear accumulation, in the hematopoietic stem cell (HSC) compartment, of somatic variants that diversely affect DNA methylation, histone modifications, pre-mRNA splicing and cell signaling (4). Clonal architecture analyzed at the single cell level indicates early clonal dominance with a very low number of residual wildtype HSCs in the bone marrow (8). Myeloid differentiation of mutated HSCs is amplified by hypersensitivity of myeloid progenitor cells to granulocyte/macrophage colony-stimulating factor (GM-CSF) (9). Virtually all the mature myeloid cells circulating in the body belong to the malignant clone (10,11).

While 15-30% of CMML patients die from disease transformation into acute leukemia, most of them demonstrate insidious physical exhaustion in an inflammatory climate. Circulating myeloid cells, predominantly monocytes and neutrophils, may contribute to the elevated levels of pro-inflammatory cytokines detected in the plasma of CMML patients (12,13).

Analysis of gene expression in peripheral blood monocytes indicates a pro-inflammatory phenotype (10,14). In mouse models of chronic myeloid malignancies, inflammatory cytokines secreted by mature myeloid cells of the leukemic clone promote HSCs expansion (15,16). Therapeutic inhibition of these cytokines prevents disease development and progression (16,17). Such feed-forward loops involving inflammatory cytokines produced by myeloid cells of the leukemic clone could contribute also to the progression of CMML, e.g., by promoting the expansion of mutated HSCs or slowing down that of wildtype cells. Yet, in chronic myeloid malignancies, the contribution of clonal mature myeloid cells and their secreted cytokines to clonal evolution remains poorly understood.

In this context, identifying new methods to block the expansion of mutated HSCs or to enhance the proliferation of wildtype cells are highly needed. The present invention fulfills this need.

### SUMMARY

In one aspect, the present invention relates to a method for treating chronic myelomonocytic leukemia (CMML), said method comprising administering to a patient in need thereof a pharmaceutical composition containing an effective amount of an inhibitor of the CXCL8 receptor.

In another aspect, the present invention relates to a method for maintaining or enhancing the proliferation of wildtype hematopoietic stem cells in patients suffering from chronic myelomonocytic leukemia (CMML), said method comprising administering to a patient in need thereof a pharmaceutical composition containing an effective amount of an inhibitor of the CXCL8 receptor.

In one aspect of the method of the invention, the CXCL8 receptor inhibitor is not used to affect tumor cell proliferation directly, but to block the CXCL8 receptor expressed by wildtype hematopoietic stem cells and the signaling pathway induced by this receptor, so as to enhance the proliferation of the wildtype hematopoietic stem cells in patients suffering from CMML, maintain their level at a sufficient level so as to slow the development of the CMML disease.

In another aspect, the present invention relates to a method for treating chronic myelomonocytic leukemia (CMML) in a subject in need thereof, said method comprising measuring the amount of immature granulocytes (IGRANs) in a sample of blood of said subject, and/or measuring the level of circulating CXCL8 in a sample of peripheral blood of said subject, and administering a pharmaceutical composition containing an effective amount of an inhibitor of the CXCL8 receptor in subjects having high number of IGRANs and/or high level of circulating CXCL8 in their blood.

### BRIEF DESCRIPTION OF THE DRAWINGS

FIG. 1. Spectral flow analysis identifies immature granulocytes (IGRANs) in CMML peripheral blood. FIG. 1A: Non-supervised UMAP of spectral flow cytometry analysis of peripheral blood cells collected from 27 CMML patients and 10 age-matched control donors.

FIG. 1B: Cell surface expression of indicated markers on the UMAP shown in A. FIG. 1C: Fraction of B, CD4+ T, CD8+ T, monocytes, dendritic cells (DC), neutrophils, and NK cells among total CD45+ cells in age-matched controls (CTL) and CMML patients. FIG. 1D: Non-supervised UMAP analysis of spectral flow cytometry data in the 10 controls compared to 27 CMML patients. FIG. 1E: Partition of neutrophil subsets based on CD15 and CD16 expression, in each group. FIG. 1F: Percentage of CD15+, CD16− and CD15+, CD16+ neutrophils as separated in E, among CD45+ cells. FIG. 1G: Correlation between CD15+CD16− and immature myeloid cell (IMC) fractions.

FIG. 2. Elevated IGRAN fraction in the peripheral blood of CMML patients is a poor prognostic factor. FIG. 2A. IGRAN fraction in CD11b+CD33+ population was measured by conventional flow cytometry in the peripheral blood of young controls (N=71), age-matched controls (N=64) and CMML patients (N=209). Kruskal-Wallis non-parametric test. FIG. 2B. IGRAN absolute number (×109/L) was measured in the peripheral blood of CMML patients compared to age-matched controls. FIG. 2C. IGRAN fraction in dysplastic (MD) and proliferative (MP) CMML subtypes according to the WHO classification. FIG. 2D. Spearman correlation between IGRAN fraction and immature myeloid cell (IMC) fraction, white blood cell count (WBC), hemoglobin level and lymphocyte fraction in the peripheral blood of CMML patients. FIG. 2E. IGRAN fraction in CMML patients grouped according to the number of mutations detected in a panel of 25 genes. Kruskal-Wallis non-parametric test. FIG. 2F. IGRAN fraction in CMML patients grouped according to the mutational status of each indicated gene: wildtype (WT) or mutated (Mut). FIG. 2G. IGRAN fraction in CMML patients grouped according to GFM, CPSS and CPSS-Molecular (CPSS-M) prognostic scores. Kruskal-Wallis non-parametric test. FIG. 2H, FIG. 2I. Event-free survival (EFS, defined as time between diagnosis and AML transformation, death, or last follow-up) (H) and overall survival (time between diagnosis and death) (I) of CMML patients with high (≥14%, N=66, lower line) or low (<14%, N=88, upper line) IGRAN fraction; log-rank test.

FIG. 3. IGRANs demonstrate features of myeloid-derived suppressive cells. FIG. 3A. Representative conventional flow plots separating IGRANs (CD45low, CD33+, CD11b+, HLA-DR−, CD14−, CD15+, CD24+, CD66b+ cells) from monocytes, PMN and lymphocytes in low density peripheral blood cells (PBMC). FIG. 3B. Single cell analysis of PBMC collected from two CMML patients with a fraction 14% and two with a lower IGRAN fraction; unsupervised clustering of pooled data separating 15 clusters groups in indicated cell categories. FIG. 3C. Dot plot showing the average expression (color-scaled) of selected granulocyte genes and the percentage of cells that expressed those genes in indicated granulocytic clusters. FIG. 3D. UMAP representation of each patient sample FIG. 3E. Fraction of each cell type in ITGAM+ clusters corresponding to CD11b+CD33+ cells per CMML patient. Colors are cluster codes defined in B. FIG. 3F. Expression of indicated genes in enriched fraction of IGRANs, monocytes and neutrophils measured by RT-qPCR and normalized to RPL32, GUS and GAPDH housekeeping genes (N=8 CMML patients). FIG. 3G. Suppressive activity of IGRANs tested in T cell proliferation assays. PBMC and CD15+-depleted PBMC (depleted-PBMC) were labeled with Cell trace Violet and proliferation of T lymphocytes upon stimulation with anti-CD3 and anti-CD28 antibodies (LT stimulation) was measured at day 4 by flow cytometry. Sorted CD15+ cells were added when indicated. N=6. FIG. 3H. Examples of dot plots showing T cell proliferation measured by cell trace violet dilution in conditions described in G.

FIG. 4. IGRANs are clonal, pro-inflammatory granulocytes. FIG. 4A. Variant allele frequency (VAF) of indicated somatic variants detected by whole exome sequencing (WES) of sorted monocytes (left) and IGRANs (right) in three CMML patients (16, 4, 10). FIG. 4B. May-Grünwald Giemsa staining of IGRAN sorted from a control donor (CTL) and a CMML patient sample, Magnification ×1000. FIG. 4C-E. Bulk RNA sequencing of sorted IGRANs collected from 7 healthy donors (CTRL) and 10 CMML patients; PCA of regularized logarithm (rlog) transformed data based on the top 500 varying genes using plotPCA function of DESeq2 package (C). MA-plot of differentially expressed genes between IGRANs collected from healthy donors and CMML patients (D). Gene set enrichment analysis (GSEA) of indicated hallmark pathways enriched in CMML vs CTL (E).

FIG. 5. CXCL8 is one of the main cytokines released by CMML patient IGRANs. FIG. 5A-D. Bulk RNA sequencing of sorted IGRAN collected from healthy donors and CMML patients. Top10 nominal enrichment score of pathways involving up-regulated genes in CMML vs CTL cells according to GO molecular function (A). Gene set enrichment analysis (GSEA) of indicated hallmark pathways enriched in CMML vs CTL (B). Gene-concept network representation of enriched genes of 4 pathways (chemokine activity in pink, chemokine receptor binding in green, cytokine activity in blue, cytokine receptor binding in violet). Dot color, log 2FoldChange. Size of beige central dots, number of core enriched genes (C). CXCL8 mRNA expression (normalized counts) in CTL cells (N=7) and CMML IGRANs (N=10)(D). FIG. 5E. CXCL8 mRNA expression assessed by RT-qPCR in healthy donor and CMML; left panels, samples used for RNA sequencing; right panel, independent cohort of IGRANs collected from 17 CTL and 18 CMML. Ct values normalized to GAPDH, RPL32 and GUS genes. FIG. 5F. Volcano plot of cytokine and chemokine levels (N=44) measured in circulating plasma of 23 IGRAN-low compared to 26 IGRAN-high CMML (threshold=14%, IGRAN fraction in box plot for each group on the left panel). FIG. 5G. Plasma levels of indicated proteins in the supernatant of IGRANs (N=7) and CMML circulating plasma compared to culture medium alone.

FIG. 6. CXCL8 specifically inhibits the growth of wildtype CD34+ cells. FIG. 6A. Cell output of CMML CD34+ cells in liquid culture for 3 days in the presence of indicated doses of CXCL8 (ng/ml); ratio to untreated samples; mean±SD, N=5 per group. FIG. 6B. Total colony output of CD34+ cells cultured in methylcellulose in the presence of indicated doses of CXCL8 (ng/ml) for 14 days. CB, cord blood; Adult, healthy donor bone marrow CD34+ cells; ratio to untreated samples; mean SD; N=6 per group. FIG. 6C. CXCR1 and CXCR2 mRNA expression assessed by RNA sequencing of CD34+ cells sorted from healthy donors (N=7) and CMML patient (N=12) bone marrow. FIG. 6D. Flow cytometry analysis of CXCR1 and CXCR2 at the surface of healthy donor (CTL, N=4) and CMML patient (N=11) CD34+ cells; left panels, fraction of cells expressing the studied receptors; right panels, mean fluorescence intensity of each receptor. FIG. 6E. Assay for Transposase-Accessible Chromatin (ATAC) sequencing experiments performed CD34+ cells obtained from two healthy donors (CTL) and 4 CMML bone marrow samples. Rectangle: peak calling for CXCR2 enhancer. FIG. 6F. Total colony output of healthy donor CD34+ culture in methylcellulose in response to CXCL8 (10 ng/mL) or CXCR1/2 inhibitor or their combination; ratio related to untreated samples; left panel, ladarixin (LADA, 10 mM), right panel, reparixin (REPA, 10 mM), mean±SD; N=7. FIG. 6G. Total colony output of healthy donor CD34+ culture in methylcellulose in the absence or presence of CXCL8 (10 ng/mL) or IGRAN supernatant or the combination of IGRAN supernatant and reparixin (REPA, 10 mM); ratio related to untreated samples; mean±SD; N=7.

FIG. 7. Spectral flow analysis of peripheral blood cells. FIG. 7A. overall survival of 580 CMML patients with IMC cutoff values at ≥1%. FIG. 7B. Cell surface marker expression in the non-supervised UMAP analysis of 37 samples (controls, 10; CMML, 27); FIG. 7C-J. Gating strategy for cell subset analysis in CD45+ cell population. Debris and doublets were discarded by gating on “Cells” (C), “Singlets” and “Singlets bis” gates (not shown). Remaining red cells were excluded and CD45+ cells were selected on a dot plot of CD45 vs SSC (D). CD15+, CD66b+ granulocytes were isolated in a “Neutro” gate (E) and separated into mature CD15+, CD16+ and immature CD15+, CD16− neutrophils (not shown). Other hematopoietic cells in a “No Neutro” gate, CD15−, CD66b− (E) include lymphoid populations, roughly isolated as expressing CD3 or CD19 or CD7 or NKG2D (LIN+) using LIN vs HLA-DR dot plot, separating B-cells (LIN+, HLA-DR+) from NK/T cells (LIN+, HLA-DR−) (F). B-cells were further detected on a CXCR4 vs CD45RA dot plot (G), NK cells on a CD56 vs CD5 dot plot (H) and T-cells as CD5+ cells, CD4+ and CD8+ T lymphocytes (I). With this exclusion gating strategy, remaining cells could be considered as monocytes and dendritic cells and monocyte subsets “Mono Pop” were separated from Dendritic cells “DC Pop” on a CD89 vs CD88 dot plot (J). FIG. 7K. Monocyte subsets in the “Mono pop” gate in the “Mono pop” gate were separated on a CD14 vs CD16 dot plot into classical, intermediate and non-classical monocytes (left panels) and the fraction of each subset was calculated in control (CTL) and CMML samples.

FIG. 8. Conventional flow detection of IGRAN in CMML peripheral blood. FIG. 8A. Representative flow plots showing the presence of IGRANs in the peripheral blood before (whole blood) and after low-density separation (N=8). FIG. 8B. Ratio of PMN (CD15+, CD16+ cells, upper panel, in grey) or IGRAN (CD15+, CD16−, lower panel, in red) to CD45+ cells before and after low density separation. N=8. Student paired test. FIG. 8C-J. Step by step gating strategy to quantify IGRAN fraction after low-density separation of peripheral blood cells; forward (FSC) and side scatter (SSC-A) are used to discard debris and dead cells (C) and doublets (D); CD45+ cells were selected on a CD45/SSC plot (E); lymphoid cells were selected with a pool of Lin antibodies (anti-CD3, CD-19 and CD-56 in the same fluorescence channel, LIN+) (F); remaining mature neutrophils were selected on a CD16/SSC plot (G); total myeloid cells were selected as CD33+, CD11b+ cells in the “LIN-/Mature neutro-” population (H); among the CD33+, CD11b+ myeloid cells, monocytes were identified as HLA-DR+ cells on a HLA-DR vs SSC plot (I); IGRANs were identified by CD15+ expression in the HLA-DR− population (J). FIG. 8K. Spearman correlation between IGRAN fraction calculated in CD11b+, CD33+ and in CD45+ populations. FIG. 8L. IGRAN fraction was serially measured, up to three times over a period of one year, in 17 CMML patients.

FIG. 9. Relationship between IGRAN fraction, CMML features and patient outcome. FIG. 9A. Spearman correlation between IGRAN fraction and patient age. FIG. 9B. IGRAN fraction in male and female CMML patients. FIG. 9C. IGRAN fraction according to the presence or absence of cytogenetic abnormalities. FIG. 9D. Spearman correlation between IGRAN fraction and mature neutrophil fraction (left panel), monocyte fraction (middle panel) and platelet count. FIG. 9E. IGRAN fraction according to WHO 2017 CMML subtypes. FIG. 9F. IGRAN fraction according to the mutational status of wildtype (WT) or mutated (Mut) status of indicated genes. FIG. 9G. Event-free survival (left) and overall survival (right) of CMML patients with high (≥0.4×109/L, N=63 in red) and low (<0.4×109/L, N=91 in blue) number of circulating IGRANs; p<0.0001, log-rank test.

FIG. 10. Molecular and functional characterization of IGRANs. FIG. 10A. Expression level of indicated genes (low expression in yellow to high expression in red) in clusters identified on the UMAP shown on FIG. 3B. FIG. 10B. Unsupervised clustering of cells in 3D (resolution 0.5 showing 15 main clusters). The dashed lined indicates ITGAM+ myeloid population selected to calculate IGRAN fraction. FIG. 10C. Spearman correlation between IGRAN and CD4+ T-cell fractions quantified by spectral flow cytometry. FIG. 10D. Proliferation assay performed in non-activated and anti-CD3/anti-CD28 activated T-cells; Forward (FSC) and side scatter (SSC-A) were used to discard debris. Dead cells and doublets were excluded using a live dead blue staining before selecting CD3+ cells using FSC/CD3 dot plot, then separating CD4+ and CD8+ T-cells.

FIG. 11. Sorted IGRAN RN sequencing and cytokines associated to their accumulation in CMML patient peripheral blood. IGRANs were sorted from healthy donor (cytapharesis, N=7) and CMML patient (N=10) peripheral blood. FIG. 11A. Mean expression of genes (using DESeq2 normalized counts) in the 17 samples sorted by decreasing order. Examples of genes highly expressed by IGRANs are in red and those highly expressed by monocytes in blue. FIG. 11B. Expression of genes (normalized counts) typically identified in IGRANs collected from controls (CTL) and CMML patients. FIG. 11C. Heatmap of sample-to-sample distances based on the rlog transformed counts. FIG. 11D. Ridgeplot representing gene set enrichment analysis (GSEA) of pathways distinguishing CMML from control IGRANs (p<0.05). FIG. 11E. Circulating levels of indicated cytokines in 23 IGRAN-Low and 26 IGRAN-High (cutoff 14%) CMML patients. FIG. 11F. Spearman correlation between IGRAN fraction in myeloid cells and plasma level of indicated cytokines.

FIG. 12. Reparixin restores CD34+ wildtype growth inhibited by CXCL8. FIG. 12A. Healthy donor bone marrow CD34+ in liquid culture were treated with 10 ng/mL CXCL8 for 72 hours; cell output compared to untreated cells. Mean ±SD, N=6. FIG. 12B. Cord blood (CB) and healthy donor bone marrow (adult) CD34+ were cultured in methylcellulose for 14 days to generate granulo-monocytic (CFU-GM, left panel) and erythroid (CFU-E, right panel) colonies in the absence or presence of indicated concentrations of CXCL8. Output of treated relative to untreated cells. Mean ±SD, N=6. FIG. 12C. Gating strategy to detect CXCR1 and CXCR2 membrane expression analysis on CD34+ cells. FIG. 12D. Spectral flow analysis of cells generated in methylcellulose for 14 days, separating on UMAP with CD45−, CD11b−, CD36+ erythroid cells and CD45+, CD11b+, CD14+ or CD45+, CD11b+, CD15+, CD66b+ granulo-monocytes (left panel), with the projection of indicated markers on UMAP (middle panel), and detecting the impact of 10 ng/mL CXCL8 on CFU-GM and on CFU-E colonies at day 14 (N=5). FIG. 12E. Cell output of healthy donor CD34+ in liquid culture of untreated and treated (CXCL8 10 ng/mL, Ladarixin 10 uM, Reparixin 10 uM and indicated combinations) healthy donor CD34+ cells in methylcellulose culture. Mean ±SD, N=6.

### DETAILED DESCRIPTION

Chronic myelomonocytic leukemia (CMML) is a severe myeloid malignancy with limited therapeutic options. The present invention explores the contribution of immature granulocytes (IGRANs) to CMML progression. IGRANs can be detected in the peripheral blood of patients by spectral and conventional flow cytometry and/or single-cell RNA sequencing. It is herein shown that these cells, whose accumulation is a potent and independent poor prognostic factor, behave as myeloid-derived suppressor cells and secrete high levels of CXCL8. Moreover, the present results highlight that this cytokine inhibits the proliferation of wildtype but not mutated hematopoietic stem and progenitor cells (HSPCs) in which CXCL8 receptors are epigenetically downregulated, and that the administration of CXCL8 receptor inhibitors such as reparixin or ladarixin restore wildtype HSPC proliferation. The relief of CXCL8 selective pressure on wildtype HSPCs in CMML patients can thus be achieved by administering CXCL8 receptor inhibitors, enabling to restore an healthy hematopoiesis and slow CMML progression.

Looking for the respective contribution of myeloid cell subsets of the leukemic clone to the inflammatory climate observed in CMML, the present inventors focused their attention on the granulocytic lineage. Neutrophil precursors, including metamyelocytes, myelocytes and promyelocytes, which are normally retained in the bone marrow, are detected cytologically in the peripheral blood of a fraction of CMML patients and referred to as immature myeloid cells (IMC) (13,18). They used single cell approaches to define immature granulocytes (IGRANs) and analyze their contribution to CMML outcome and pathogenesis. Their results show that, in two independent cohorts of patients, the presence of immature granulocytes in the peripheral blood correlates with a poor outcome. They also show that these IGRANs cells, which share the same clonal origin as monocytes, demonstrate an inflammatory and immunosuppressive phenotype, secreting high levels of CXCL8. Finally, they show here that the lack of response of clonal HSPCs to CXCL8 is due to the downregulation of the CXCL8 receptors CXCR1 and CXCR2 at the surface of these cells as consequence of clonal epigenetic changes.

The present disclosure shows here for the first time that the IL-8/CXCL8 cytokine secreted by IGRANs inhibits the proliferation and differentiation of wildtype HSCs while sparing mutated HSCs in which CXCL8 receptors are downregulated as consequence of clonal epigenetic changes. In view of these surprising results, they hypothesized that it would be possible to slow CMML progression by blocking CXCL8, its receptor, or its signaling pathway.

In this context, the present inventors used two known CXCL8 receptors inhibitors, Ladarixin and Reparixin, targeting the CXCL8-CXCR1/2 axis, to see if they could restore the proliferation of wild-type CD34+ cells in the presence of CXCL8. They showed that the use of each of the two pharmacological agents targeting the CXCL8 receptor indeed restores the proliferation of wildtype HSCs, even in the presence of CXCL8. Thus, the feed-forward loop that involves CXCL8 produced by immature granulocytes of CMML leukemic clone in the repression of residual wildtype HSC unravels a new strategy for the therapeutic management of CMML patients, to restore wild-type HSCs and slow disease progression.

Thus, the present invention relates to a method for treating chronic myelomonocytic leukemia (CMML), said method comprising administering to a patient in need thereof a pharmaceutical composition containing an effective amount of an inhibitor of the CXCL8 receptor. The present invention also relates to a method for maintaining or enhancing the proliferation of wildtype hematopoietic stem cells in patients suffering from chronic myelomonocytic leukemia (CMML), said method comprising administering to a patient in need thereof a pharmaceutical composition containing an effective amount of an inhibitor of the CXCL8 receptor. In another aspect of the method of the invention, the CXCL8 receptor inhibitor is not used to affect tumor cell proliferation directly, but to block the CXCL8 receptor expressed by wildtype hematopoietic stem cells and the signaling pathway induced by this receptor, so as to enhance the proliferation of the wildtype hematopoietic stem cells in patients suffering from CMML, maintain their level at a sufficient level so as to slow the development of the CMML disease.

### The CXCL8 Receptor

Interleukin 8 (IL-8 or chemokine (C—X—C motif) ligand 8, or CXCL8) is a chemokine also known as NAF; GCP1; LECT; LUCT; NAP1; GCP-1; LYNAP; MDNCF; MONAP; NAP-1; or SCYB8. In humans, the CXCL8 protein is encoded by the CXCL8 gene (Gene ID: 3576). The protein encoded by this gene is a member of the CXC chemokine family and is a major mediator of the inflammatory response. The encoded protein is commonly referred to as interleukin-8 (IL-8) or CXCL8. It is secreted by mononuclear macrophages, neutrophils, eosinophils, T lymphocytes, epithelial cells, and fibroblasts. It functions as a chemotactic factor by guiding the neutrophils to the site of infection. This protein is also secreted by tumor cells and promotes tumor migration, invasion, angiogenesis and metastasis.

CXCL8 is initially produced as a precursor peptide of 99 amino acids which then undergoes cleavage to create several active CXCL8 isoforms. In humans, there are two precursors referenced as NP_000575.1 (99 aa, generating the isoform 1) and NP_001341769.1 (95 aa, generating the isoform 2).

There are many receptors on the surface membrane capable of binding CXCL8; the most frequently studied types are the G protein-coupled serpentine receptors CXCR1 and CXCR2. Expression and affinity for CXCL8 differs between the two receptors (CXCR1>CXCR2).

CXCR1 is a member of the G-protein-coupled receptor family also known as chemokine (C—X—C motif) receptor 1 (CXC-R1, CXCR-1), CD128; CD181; CKR-1; IL-8 receptor type 1 (IL8R1); IL-8 receptor type A or alpha (IL-8RA); CMKAR1; IL8RBA; CDw128a; or C-C-CKR-1. In humans, the CXCR1 protein is encoded by the CXCR1 gene (Gene ID: 3577). In humans, it has the sequence referenced as NP_000625.1 (350 amino acids).

CXCR2 is a member of the G-protein-coupled receptor family also known as chemokine (C—X—C motif) receptor 2 (CXC-R2, CXCR-2), IL-8 receptor type 2 (IL-8R2) or beta (IL-8RB); CD182, CDw128b, CMKAR2, or WHIMS2. In humans, the CXCR2 protein is encoded by the CXCR2 gene (Gene ID: 3579). In humans, it has the sequence referenced as NP_001548.1 (360 amino acids). Several isoforms are known.

### CXCL8 Receptor Inhibitors

CXCL8 receptor inhibitors used in the methods of the invention preferably inhibit binding of CXCL8 to its receptors and/or the intracellular signaling activated by the binding of CXCL8 to its receptors. As explained above, these receptors are CXCR1 and CXCR2. The CXCL8 receptor inhibitor used in the methods of the invention can be either allosteric inhibitors or orthosteric antagonists of CXCR1 receptor or of CXCR2 receptor or of both CXCR1 and CXCR2 receptors as defined above.

According to one embodiment, the CXCL8 receptor inhibitor used in the methods of the invention inhibits the binding of CXCL8 to CXCR1 receptor and/or the intracellular signaling activated by the binding of CXCL8 on the CXCR1 receptor. Preferably, said CXCL8 receptor inhibitor is either an allosteric inhibitor or an orthosteric antagonist of the CXCR1 receptor. In a preferred embodiment, the CXCL8 receptor inhibitor used in the methods of the invention has an IC₅₀ value towards CXCR1 receptor in the low nanomolar range, preferably in the range 0.02-5 nanomolar.

According to an alternative embodiment, the CXCL8 receptor inhibitor used in the methods of the invention inhibits the activity of CXCL8 mediated by CXCR2 receptor. Preferably, according to this embodiment, said CXCL8 receptor inhibitor inhibits the binding of CXCL8 to CXCR2 receptor and/or the intracellular signaling activated by the binding of CXCL8 on the CXCR2 receptor. Preferably, said CXCL8 receptor inhibitor is either an allosteric inhibitor or an orthosteric antagonist of the CXCR2 receptor. More preferably, the CXCL8 receptor inhibitor used in the methods of the invention has an IC₅₀ value towards CXCR2 receptor in the low nanomolar range, preferably in the range 0.02-5 nanomolar. Preferred CXCL8 receptor inhibitors according to this embodiment are those disclosed in WO2007135080, such as the 2-{4[(isopropylsulfonyl)amino]phenyl}propanamide, preferably the (2R)-2-{4[(isopropylsulfonyl)amino]phenyl}propanamide, or pharmaceutically acceptable salts thereof, preferably the sodium salt thereof.

Preferably, the CXCL8 receptor inhibitor used in the methods of the invention is selected from small molecular weight molecules, peptides and antibodies.

Preferably, the CXCL8 receptor inhibitor used in the methods of the invention is selected from:
the anti-CXCL-8 antibodies ABCream, BMS-986253 and ABX-IL-8;
RP-72, PAC-G-31-P, SCH-N;
Navarixin, having formula

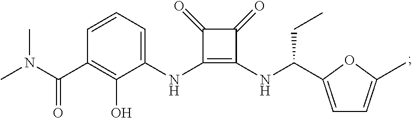

SX-517, having formula

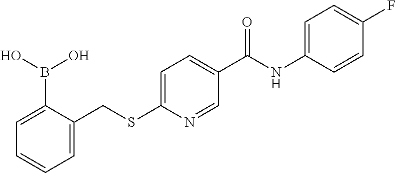 [SMILES: O=C(Nc1ccc(F)cc1)c1ccc(SCc2ccccc2B(O)O)nc1]

SX-576, having formula:

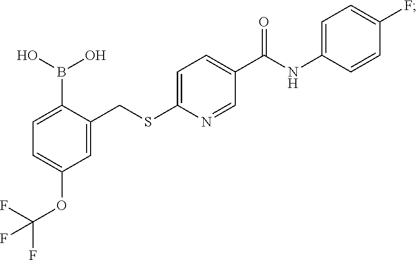 [SMILES: O=C(Nc1ccc(F)cc1)c1ccc(SCc2cc(OC(F)(F)F)ccc2B(O)O)nc1]

SX-682, having formula:

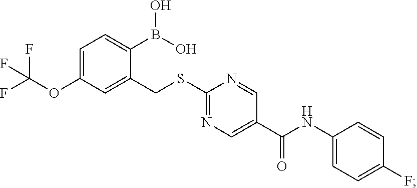 [SMILES: O=C(Nc1ccc(F)cc1)c1cnc(SCc2cc(OC(F)(F)F)ccc2B(O)O)nc1]

the compounds having formula:

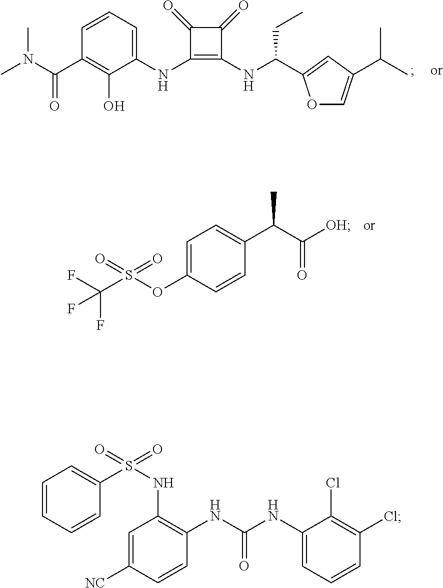 [SMILES: CC[C@@H](Nc1c(Nc2cccc(C(=O)N(C)C)c2O)c(=O)c1=O)c1cc(C(C)C)co1 ; C[C@@H](C(=O)O)c1ccc(OS(=O)(=O)C(F)(F)F)cc1 ; N#Cc1ccc(NC(=O)Nc2cccc(Cl)c2Cl)c(NS(=O)(=O)c2ccccc2)c1]

5-[3-(2-Fluorophenyl)ureido]-1-(2-hydroxypropyl)-1H-pyrazole-4-carboxylic acid ethyl ester;
5-[3-(3-Fluorophenyl)ureido]-1-(2-hydroxypropyl)-1H-pyrazole-4-carboxylic acid ethyl ester;
3-[2-[1(R)-(4-Bromofuran-2-yl)propylamino]-3,4-dioxo-1-cyclobutenylamino]-2-hydroxy-N,N-dimethylbenzamide;
3-[2-[1(R)-(4-Chlorofuran-2-yl)propylamino]-3,4-dioxo-1-cyclobutenylamino]-2-hydroxy-N,N-dimethylbenzamide;
Trifluoromethanesulfonic acid 4-[1(R)-(N-isopropylcarbamoyl)ethyl]phenyl ester;
2-Hydroxy-3-[4-[1(R)-(4-isopropylfuran-2-yl)propylamino]-1-oxo-1,2,5-thiadiazol-3-ylamino]-N,N-dimethylbenzamide;
3-(2-Chlorophenylamino)-7-nitro-4H-1,2,4-benzothiadiazin-5-ol 1,1-dioxide;
1-[3-[4-[3-(4-Fluorophenyl)isoxazol-5-yl]phenoxy]propyl]-4-methylpiperazine;
N-(2-[(2,3-Difluorobenzyl)sulfanyl]-6-[[(2R,3S)-3,4-dihydroxybutan-2-yl]oxy]pyrimidin-4-yl)azetidine-1-sulfonamide;
   the CXCL8 receptor(s) inhibitors of formula (I), also described in WO2000/024710 and WO2005/090295:

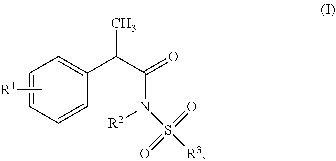

wherein
R¹ is selected from a linear or branched C₁-C₆ alkyl, benzoyl, phenoxy, and trifluoromethanesulfonyloxy (preferably, R1 is selected from benzoyl, isobutyl, and trifluoromethanesulfonyloxy; more preferably, R1 is linked to the phenyl ring in 3- or 4-position; even more preferably, R1 is 3-benzoyl, 4-isobutyl or 4-trifluoromethanesulfonyloxy,
R² is selected from hydrogen atom and linear or branched C₁-C₃ alkyl; preferably, R² is selected from hydrogen atom or methyl,
R³ is a linear or branched C₁-C₆ alkyl or trifluoromethyl; R³ is preferably selected from linear or branched C₁-C₆ alkyl, more preferably from linear of branched C₁-C₃ alkyl, even more preferably, R³ is methyl,
or a pharmaceutically acceptable salt thereof;
the CXCL8 receptor(s) inhibitors of the following formula (II), described in WO2010/031835:

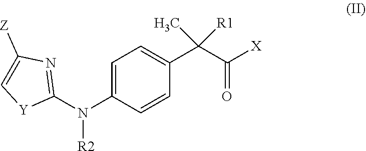

wherein:
R1 is hydrogen,
X is OH,
R2 is hydrogen or linear C₁-C₄alkyl,
Y is a heteroatom selected from S, O and N,
Z is selected from linear or branched C₁-C₄ alkyl, linear or branched C₁-C₄ alkoxy, halo C₁-C₃ alkyl and halo C₁-C₃ alkoxy,
and pharmaceutically acceptable salts thereof.

Preferably, the chiral carbon of the compounds of formula (I) is in the RS or R configuration, more preferably it is in the R configuration.

Particularly preferred among said CXCL8 inhibitors of formula (I) are:
2-(4-isobutylphenyl)propionyl methansulfonamide, preferably R-(−)-2-(4-isobutylphenyl)propionyl methansulfonamide and pharmaceutically acceptable salts thereof, preferably the lysine salt thereof (also known as reparixin), and
2-(4-trifluoromethanesulfonyloxy)phenyl]-N-methanesulfonyl propionamide, preferably R(−)-2-(4-trifluoromethanesulfonyloxy)phenyl]-N-methanesulfonyl propionamide and pharmaceutically acceptable salts thereof, in particular the sodium salt thereof (also known as ladarixin or DF2156A).

Preferably, the chiral carbon of the compounds of formula (II) is in the RS or S configuration, more preferably in the S configuration.

Particularly preferred among said CXCL8 inhibitors of formula (II) are 2-(4-{[4-(trifluoromethyl)-1,3-thiazol-2-yl]amino}phenyl)propanoic acid, preferably (2S)-2-(4-{[4-(trifluoromethyl)-1,3-thiazol-2-yl]amino}phenyl) propanoic acid or the sodium salt thereof, and 2-methyl-2-(4-{[4-(trifluoromethyl)-1,3-thiazol-2-yl]amino}phenyl)propanoic acid (DF2726Y) and pharmaceutically acceptable salts thereof, preferably the sodium salt (DF2726A).

More preferably, the CXCL8 receptor(s) inhibitor used in the methods of the invention can be chosen from: AZD5069, SX-682, SX-576, SX-517, navarixin, ladarixin, reparixin, reparixin L-lysine salt, DF2755A, the CXCL8 fragment comprising amino acids 3-74 and substitutions K11R/G31P (G31P), DF2162, and SCH-479833, or combinations thereof. It can also be a neutralizing antibody that binds specifically to human IL-8, such as the human anti-IL-8 antibodies ABCream, ABX-IL8 or HuMax-IL8 (now known as BMS-986253) which are human monoclonal antibodies that inhibit interleukin-8 (IL-8).

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism. Preferably, the pharmaceutical composition of the present invention is prepared in suitable dosage forms comprising an effective amount of a CXCL8 receptor inhibitor, a salt thereof with a pharmaceutically acceptable organic or inorganic acid or base, or a prodrug thereof, and at least one inert pharmaceutically acceptable excipient.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, and the patient's disposition to the disease and the judgment of the treating physician.

### Reparixin

The most preferred CXCL8 receptor inhibitor useful in the methods of the present invention is 2-(4-isobutylphenyl)propionyl methansulfonamide, specifically the R-(−)-2-(4-isobutylphenyl)propionyl methansulfonamide (reparixin) or pharmaceutically acceptable salts thereof, preferably the lysine salt thereof (also known as reparixin L-Lysine salt, PubChem CID 9932389).

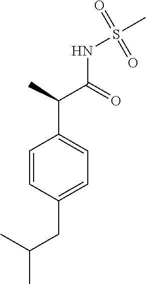 [SMILES: CC(C)Cc1ccc([C@@H](C)C(=O)NS(C)(=O)=O)cc1]

(PubChem CID 9838712; repertaxin, DF1681B)

Reparixin (formerly known as repertaxin) is a noncompetitive metastable inhibitor of CXCR1 and CXCR2 with a 400-fold greater potency to inhibit CXCR1 activity than CXCR2 (53). It was originally evaluated as a drug to prevent graft rejection for pancreatic islet cells. Clinical studies on reparixin have been completed evaluating its use for the treatment of ischemia-reperfusion injury, organ transplant-associated disease. The results showed that reparixin can effectively regulate neutrophil recruitment and attenuate inflammatory injury in ischemia-reperfusion (54).

In vitro studies with reparixin in thyroid cancer found that it also exhibits direct anti-tumor activity by inhibiting tumor cell proliferation (55). In a phase I clinical trial in patients with HER-2 negative metastatic breast cancer, reparixin was well tolerated in combination with paclitaxel chemotherapy (56). However, phase II double blinded clinical trials in triple negative breast cancer patients demonstrated no improvement reparixin in combination with paclitaxel exhibited no additional clinical benefit compared to treatment with paclitaxel alone (cf. the clinical trial NCT02370238 and 57).

According to a preferred embodiment, when the CXCL8 inhibitor is R-(−)-2-(4-isobutylphenyl)propionyl methansulfonamide, it can be administered to said patient either intravenously or orally. If administered intravenously, it may be at a dose between 2 and 3 mg/kg body weight/hour, preferably of 2.772 mg/kg body weight/hour, for 7 days.

[claim 7] If administered to said patient orally, it may be at a dosage of 400, 800, and 1200 mg three times a day, preferably at a dosage of 1200 mg three times a day.

### Ladarixin

Ladarixin (DF 2156A free base) is an orally active, allosteric non-competitive and dual CXCR1 and CXCR2 antagonist. It has the following formula:

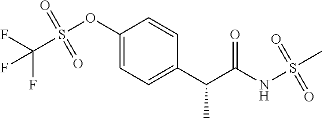 [SMILES: C[C@@H](C(=O)NS(C)(=O)=O)c1ccc(OS(=O)(=O)C(F)(F)F)cc1]

4-[(2 R)-1-oxo-1-(methanesulfonami do)propan-2-yl]phenyl trifluoromethanesulfonate

CXCR1 and CXCR2 inhibition by Ladarixin improves neutrophil-dependent airway inflammation in mice (58). Also, preclinical evaluation of ladarixin demonstrated significant activity in a mouse model of pancreatic ductal adenocarcinoma (PDAC): the combination of ladarixin and an anti-PD-1 monoclonal antibody showed improved antitumor activity compared to either agent alone (59). In an animal model of uveal melanoma administration of ladarixin repolarized TAMs to a M1 phenotype and inhibited tumor cell migration. Ladarixin has been used in clinical trials for diabetes, however clinical trials for cancers have not been reported (57). The dose is 400 mg twice a day.

Therapeutic Methods with a Prognostic Step

The results of the present inventors identify IGRAN excess quantified by flow cytometry as a novel and independent prognostic factor in CMML. CXCL8 secreted by these cells that belong to the leukemic clone specifically inhibit wildtype CD34⁺ cells, giving a competitive advantage to CMML-mutated cells that have lost CXCL8 receptor expression. The inventors therefore propose to relieve CXCL8 selective pressure on wildtype HSPCs, by administering an inhibitor of CXC chemokine receptor types 1 (CXCR1) and 2 (CXCR2), in order to modulate clonal evolution and reduce the dominance of the mutated clone, in patients suffering from CMML having a high level of IGRANs in their blood.

More precisely, it is herein shown that flow cytometry quantification of IGRANs among circulating myeloid cells provides an independent prognostic information. Flow cytometry analysis of peripheral blood cells, which supports CMML diagnosis by identifying an abnormal partition of monocyte subsets (2, 21, 22), is shown here to be also as a powerful stratification tool by quantifying the IGRAN fraction or their absolute number.

The present inventors propose a precise and reproducible measurement of IGRAN fraction by flow cytometry identification of CD45^{low}, CD11b⁺, CD14⁻, CD15⁺, CD16⁻, CD24⁺, CD33⁺, CD66b⁺, HLA-DR⁻ cells in the peripheral blood, with cutoff values at 14% of CD11b⁺CD33⁺ cells or 0.4×10⁹/L.

In a preferred embodiment, the methods of the invention comprise an additional step of measuring the amount of circulating IGRANs in the blood of the patients, before administering the pharmaceutical composition of the invention. In this case, the present invention relates to a method for treating chronic myelomonocytic leukemia (CMML) in a subject in need thereof, said method comprising:
(a) measuring the amount of Immature granulocytes (IGRANs) in a sample of blood of said subject,
(b) administering a pharmaceutical composition containing an effective amount of an inhibitor of the CXCL8 receptor in subjects having high number of IGRANs in their blood.

Step a) more generally relates to detecting and quantifying the quantity, density or frequency of immature granulocytes (IGRANs) circulating in the blood of the subjects. This can be performed by conventional flow cytometry or by single-cell RNA sequencing.

Flow cytometry is a powerful technology that allows researchers and clinicians to perform complex cellular analysis quickly and efficiently by analysing several parameters simultaneously. The amount of information obtained from a single sample can be further expanded by using multiple fluorescent reagents. The information gathered by the flow cytometer can be displayed as any combination of parameters chosen by the skilled person. Cells pass single-file through a laser beam. As each cell passes through the laser beam, the cytometer records how the cell or particle scatters incident laser light and emits fluorescence. Using a flow cytometric analysis protocol, one can perform a simultaneous analysis of surface molecules at the single-cell level.

More preferably, the detection of the cell surface antigens in the methods of the invention is performed by an exclusion gating strategy by flow cytometry.

In a preferred embodiment, the measurement of IGRANs is performed in step a) by flow cytometry identification of CD45^{low}, CD11b⁺, CD14⁻, CD15⁺, CD16⁻, CD24⁺, CD33⁺, CD66b⁺, HLA-DR− cells in the peripheral blood of said subject.

In a more preferred embodiment, the measurement of IGRANs is performed in step a) by flow cytometry identification of CD45^{low} CD15⁺CD16⁻ and CD66b⁺ cells, preferably of CD45^{low}, CD11b⁺, CD14⁻, CD15⁺, CD16⁻, CD24⁺, CD33⁺, CD66b⁺, HLA-DR⁻ cells in the peripheral blood. Preferably, cutoff values at 14% of CD11b⁺CD33⁺ cells or 0.4×10⁹/L are applied.

In these particular methods, the pharmaceutical composition containing an effective amount of an inhibitor of the CXCL8 receptor is administered in subjects having a high number of IGRANs in their blood.

By “high number of IGRANs”, it is herein meant that the proportion of IGRANs in the PBMCs of the tested subject is significantly higher than a reference value. Said reference value can be for example the proportion of IGRANs in a PBMC reference sample obtained from a patient that does not suffer from CMML, preferably from an healthy subject. By “significantly higher”, it is herein meant that the proportion of IGRANs among PBMC in the sample of the tested subject is twice, or three times, four times, five times, or ten times more than the reference value obtained from the reference sample(s).

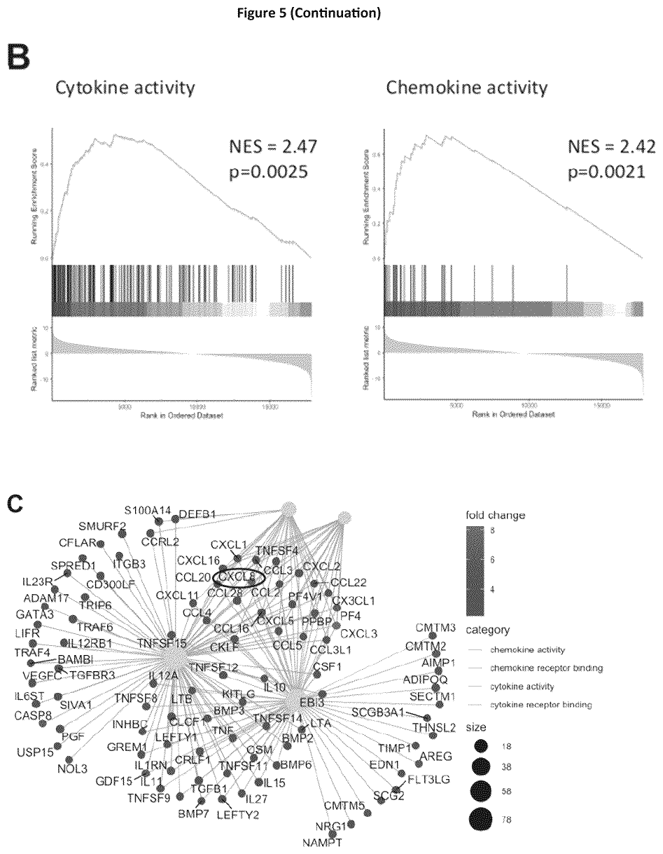

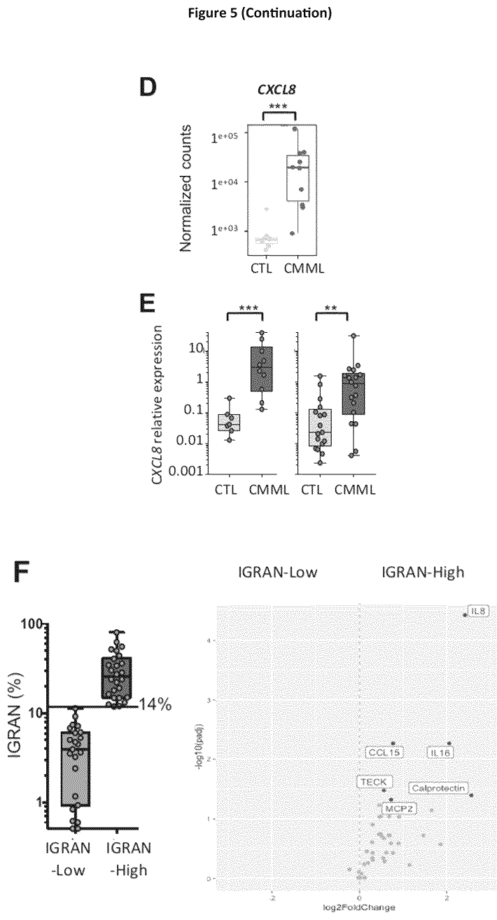

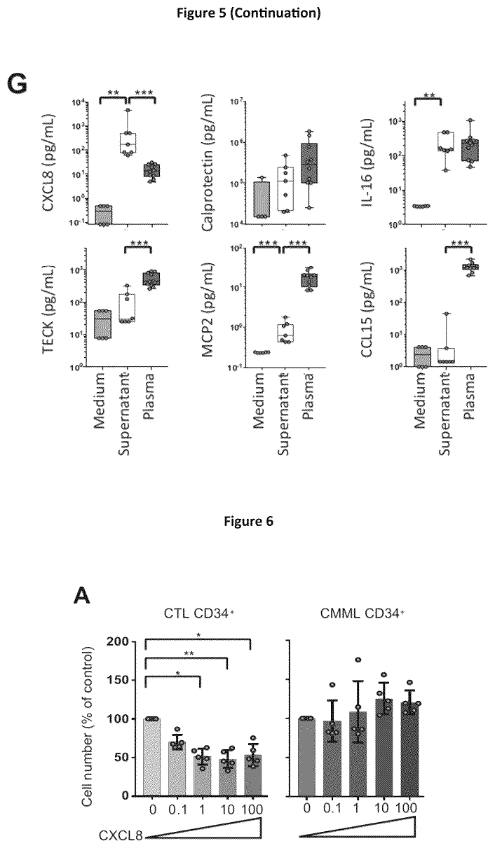

Comparison of sorted CMML IGRANs with immature granulocytes collected by cytapheresis from the peripheral blood of healthy donors after mobilization, suggested a typical combination of cytokine gene expression in CMML cells. Previous studies had detected high levels of multiple cytokines, including TNFα, IL-1β, IL-6, and CXCL8, in the circulating plasma and bone marrow supernatant of CMML patients, leading to heterogeneous patterns of inflammatory protein levels (12). To date, these patterns have failed to predict the clinical response to therapeutic agents such as ruxolitinib (44). Here, six cytokines were found to be overproduced in the peripheral blood of patients in the IGRAN-high CMML group, of which CXCL8 was at the highest levels, showed a direct correlation with IGRAN quantification, and was detected in IGRAN supernatant (FIGS. 5C-F). These results suggest that in the heterogeneous population of CMML patients, combining IGRAN quantification by flow cytometry with circulating CXCL8 level defines a subgroup of patients most likely to benefit from a therapeutic strategy targeting the CXCL8-mediated pathway.

In a particular embodiment, it is also possible to measure circulating CXCL8 level independently of IGRAN quantification.

In a preferred embodiment, the methods of the invention therefore also comprise a step of measuring the level of circulating CXCL8 in the blood or in the plasma of the subject. This measurement can be performed by any conventional means, e.g., by flow cytometry, RT-PCR, ELISA, western blot, etc.

This step can be performed in addition to the IGRAN quantification, or instead of IGRAN quantification (i.e., in combination with or alternatively).

In this case, in the methods of the invention, the pharmaceutical composition containing an effective amount of an inhibitor of the CXCL8 receptor is administered in subjects having a high level of circulating CXCL8 in their blood.

By “high level of CXCL8”, it is herein meant that the amount of CXCL8 in the blood or plasma of the tested subject is significantly higher than a reference value. Said reference value can be for example the amount of CXCL8 in a blood or plasma reference sample obtained from a patient that does not suffer from CMML, preferably from an healthy subject. By “significantly higher”, it is herein meant that the amount of CXCL8 in the sample of the tested subject is between two and hundred times more than the reference value obtained from the reference sample(s), depending on the measurement system used for detecting CXCL8.

### Combination Treatment

The therapeutic methods of the invention can be advantageously combined with other therapeutic strategies to overcome the suppressive potential of IGRANs.

In particular, these methods can further include a step of reducing granulocytic dysplasia with hypomethylating agents or controlling cell proliferation through JAK-STAT pathway or GM-CSF inhibition. This step can be achieved by administering to the subject a further active agent, for example an hypomethylating agent (such as azacitidine, decitabine, decitabine-ceduzaridine) or a JAK-STAT inhibitor (such as ruxolitinib, fedratinib) or a GM-CSF inhibitor (such as lenzilumab).

Moreover, the methods of the invention can also comprise a further step of administering, together with or separately of the pharmaceutical composition of the invention containing a CXCL8 receptor inhibitor, a direct tumorigenic agent, for example a chemotherapeutic agent, that will affect the proliferation of the mutated HSC present in the blood of the CMML patients.

Said direct tumorigenic agent can be any compound that is known to be able to affect the proliferation of the mutated HSC present in the blood of the CMML patients. In a particular embodiment, said direct tumorigenic agent is chosen from: topotecan, hydroxyurea, anthracyclines-Ara C, cytarabine, bortezomib, farnesyl transferase inhibitors, histone deacetylase inhibitors, arsenic trioxide, and DNA methyltransferase inhibitors, such as 5-azacitidine, 5-aza-2′-deoxyazacytidine, or decitabine.

### Definitions

A “subject” which may be subjected to the methodology described herein may be any of mammalian animals including human, dog, cat, cattle, goat, pig, swine, sheep and monkey. More preferably, the subject of the invention is human subject; a human subject can be known as a patient. In one embodiment, “subject” or “subject in need” refers to a mammal, preferably a human, that suffers from CMML or is suspected of suffering from CMML or has been diagnosed with CMML. As used herein, a “CMML suffering subject” refers to a mammal, preferably a human, that suffers from CMML or has been diagnosed with CMML. A “control subject” refers to a mammal, preferably a human, which is not suffering from CMML, and is not suspected of being diagnosed with CMML.

The “frequency” of a particular cell population in a given sample is herein understood as being the proportion of this particular cell population among the cells present in said sample. It can be measured by calculating the percentage of cells of this particular population (i.e., displaying the markers known to be shared by the cells of this population) present in said sample, among the total number of cells present in the tested sample, or among the cells of another particular cell population (e.g., in the context of the invention, among all DC cells or among CD45+ leukocytes). It can also be the number of cells belonging to the target population divided by the number of other cells, provided that said number of other cells is normalized between samples, so as to be comparable. In that sense, the “frequency” of the cells of the invention can be herein assimilated to the “concentration” or the “abundance” of the cells belonging to the target population of the invention, within a particular category of cells. The term “frequency” as meant herein is therefore synonymous of the terms “proportion”, “percentage” or “concentration” which can be used interchangeably.

The term “reference value”, as used herein, refers to the expression level of a prognosis marker under consideration in a reference sample. The suitable reference expression levels can be determined by measuring the expression levels of said prognosis marker in several suitable subjects, and such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. It can also be based on a sample from the subject being tested, taken from a non-cancerous tissue (i.e., a normal tissue of the same subject). The reference value can be based on a large number of samples, such as from population of subjects of the chronological age matched group, or based on a pool of samples including or excluding the sample to be tested.

As used herein, the terms “treat”, “treating”, “treatment”, and the like refer to reducing or ameliorating the symptoms of a disorder (e.g., CMML), and/or symptoms associated therewith. It will be appreciated that, although not precluded, treating a disorder or condition does not require that the disorder, condition or symptoms associated therewith be completely eliminated.

As used herein “treating” a disease in a subject or “treating” a subject having a disease refers to subjecting the subject to a pharmaceutical treatment, e.g., the administration of a drug, such that the extent of the disease is decreased or prevented. For examples, treating results in the reduction of at least one sign or symptom of the disease or condition. Treatment includes (but is not limited to) administration of a composition, such as a pharmaceutical composition, and may be performed either prophylactically, or subsequent or the initiation of a pathologic event. Treatment can require administration of an agent and/or treatment more than once.

“Prognosis” herein means the prediction/determination/assessment of the risk of disease (in particular a cancer and/or a tumour) progression (or evolution, or development) in an individual. Prognosis includes the assessment of the future development of the subject's condition and the possible chances of cure. The prognosis can be determined on the basis of observations and/or measurements, carried out using various tools.

As used herein, the term “active ingredient” refers to the agent accountable for the intended biological effect (e.g., CXCR1/2 inhibitor). Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols. Techniques for formulation and administration of drugs may be found in the latest edition of “Remington's Pharmaceutical Sciences,” Mack Publishing Co., Easton, PA, which is herein fully incorporated by reference.

In the present description and in the following claims, the wording “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. The amount of a CXCL8 receptor inhibitor or the pharmaceutically acceptable salt thereof in the pharmaceutical composition of the present invention can vary over a wide range depending on known factors, for example, the molecule used, the severity of the disease, the patient's body weight, the dosage form, the chosen route of administration, and the number of administrations per day. However, a person skilled in the art can determine the optimum amount in easily and routinely manner.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, intraperitoneal, intranasal, or intraocular injections. Alternately, one may administer the pharmaceutical composition in a local rather than systemic manner, for example, via injection of the pharmaceutical composition directly into a tissue region of a patient. Pharmaceutical compositions of the present invention may be manufactured by processes well known in the art, e.g., by means of conventional mixing, dissolving, granulating, dragee making, levigating, emulsifying, encapsulating, entrapping, or lyophilizing processes. Preferably, the pharmaceutical composition is for intravenous or oral administration.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The CXCL8 receptor inhibitor compounds of the present invention may form stable pharmaceutically acceptable acid or base salts with a pharmaceutically acceptable organic or inorganic acid or base, and in such cases administration of a compound as a salt may be appropriate.

Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate.

Examples of base addition salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Nontoxic physiologically-acceptable salts are preferred, although other salts may be useful, such as in isolating or purifying the product.

The salts may be formed by conventional means, such as by reacting the free form of the product with one or more equivalents of the appropriate acid or base in a solvent or medium in which the salt is insoluble, such as for example water or ethanol, which is removed under vacuum or by freeze drying.

The present invention also includes the prodrugs, stereoisomers, isotope-labelled, for example deuterated, derivatives and enantiomers of the CXCL8 inhibitor compounds described above. As used herein the term “prodrug” refers to an agent, which is converted into the parent drug in vivo by some physiological chemical process (e.g., a prodrug on being brought to the physiological pH is converted to the desired drug form). Prodrugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a prodrug would be a compound of the present invention wherein it is administered as an ester (the “prodrug”) to facilitate transmittal across a cell membrane where water solubility is not beneficial, but then it is metabolically hydrolysed once inside the cell where water solubility is beneficial. Prodrugs have many useful properties. For example, a prodrug may be more watersoluble than the ultimate drug, thereby facilitating intravenous administration of the drug. A prodrug may also have a higher level of oral bioavailability than the ultimate drug. After administration, the prodrug is enzymatically or chemically cleaved to deliver the ultimate drug in the blood or tissue. Ester prodrugs of the CXCL8 inhibitor compounds disclosed herein are specifically contemplated. While not intending to be limiting, an ester may be an alkyl ester, an aryl ester, or a heteroaryl ester. The term alkyl has the meaning generally understood by those skilled in the art and refers to linear, branched, or cyclic alkyl moieties. C1-6 alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1 to 6 carbon atoms. Certain CXCL8 inhibitor compounds may exist in tautomeric forms, and this invention includes all such tautomeric forms of those compounds unless otherwise specified.

Unless otherwise stated, structures depicted herein are also meant to include all stereochemical forms of the structure; i.e., the R and S configurations for each asymmetric centre. Thus, single stereochemical isomers as well as enantiomeric and diastereomeric mixtures of the present compounds are within the scope of the invention. Thus, this invention encompasses each diastereomer or enantiomer substantially free of other isomers (>90%, and preferably >95%, free from other stereoisomers on a molar basis) as well as a mixture of such isomers. Particular optical isomers can be obtained by resolution of the racemic mixtures according to conventional processes, e.g., by formation of diastereomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereomers by crystallization followed by liberation of the optically active bases from these salts. A different process for separation of optical isomers involves the use of a chiral chromatography column optimally chosen to maximize the separation of the enantiomers. Still another method involves synthesis of covalent diastereomers by reacting compounds of the invention with an optically pure acid in an activated form or an optically pure isocyanate. The synthesized diastereomers can be separated by conventional means such as chromatography, distillation, crystallization or sublimation, and then hydrolysed to deliver the enantiomerically pure compound. Optically active compounds of the invention can be obtained by using active starting materials. These isomers may be in the form of a free acid, a free base, an ester or a salt.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The following examples serve to illustrate certain aspects of the disclosure and should not be construed as limiting the claims.

### EXAMPLES

### Material and Methods

Heath donor and patient samples. Peripheral blood (PB) samples were collected with institutional review board approbations before any treatment from patients with a diagnosis of CMML according to the WHO classification. PB films from 580 CMML collected from Mayo clinic (Rochester, MN, USA) were evaluated for immature myeloid cells (IMC [myelocytes, metamyelocytes, promyelocytes]≥1%) (19). CMML samples for validation cohort were collected between March 2015 and April 2019 from eight French centers. Control samples were routine tube remnants (age ≥65-years, Henri Mondor Hospital Créteil, France), remnants from cytapheresis in stem cell donors (Gustave Roussy, Villejuif, France) and buffy coats from blood donors (Age <65-years, Etablissement Francçais du Sang, Rungis, France). Bone marrow CD34⁺ cells from adult healthy donors were obtained from Lonza laboratories and the bone bank of Cochin hospital, and umbilical cord blood samples were collected from Saint-Louis hospital (AC-2016-2759, Paris).

Cell sorting. PB samples collected on EDTA were processed within 24 hours. When indicated, Blood Cell Stabilizer (Cytodelics, Cytodelics AB) was mixed at a 1:1 ratio to 1 mL of whole blood and transferred to −80° C. freezer. In other cases, samples were centrifuged at 300 g for 5 min at room temperature (RT), plasma was collected, then peripheral blood mononuclear cells (PBMC) were isolated using Pancoll density centrifugation (Pan-Biotech, Dutscher). CD16⁺ neutrophils were sorted from the white cell layer directly above the red blood cells using immuno-magnetic microbeads (AutoMacs system, Miltenyi Biotech). PBMC were used for conventional flow cytometry or immuno-magnetic sorting of CD3⁺ T-cells, CD14⁺ monocytes or IGRANs (Classical Monocyte Cocktail, Miltenyi Biotec). Sorted cells (purity ≥90%) were stored at −80° C. as dry pellets. IGRANs were centrifuged on microscope slides, dried for 1 hour at RT, and stained with May-Grünwald-Giemsa. Patient CD14⁺ DNA were subjected to next generation sequencing (NGS) for a myeloid panel (3). CD34⁺ cells were sorted by AutoMacs system and frozen in FBS-DMSO 10%.

Spectral and conventional flow cytometry. Cryopreserved PB samples were thawed at 37° C. and fixed before red blood cell lysis, as recommended by the manufacturer. Cells were washed and incubated with antibodies for 1 hour at 4° C., washed in BSA 1%/EDTA 0.5M in PBS and analyzed on CyTEK Aurora flow cytometer (Cytek Biosciences). FCS files were exported using FlowJO software. Marker expression values were transformed using the auto-logicle transformation function. Phenograph clustering was performed using 28 markers and a number of nearest neighbors of 30. UMAP was run with a nearest neighbor of 15 and a min_distance of 0.2. Conventional flow cytometry analysis was performed on 200 μL of whole blood cells using a lyse no wash protocol (Versalyse lysing solution, Beckman Coulter) or on 2×10⁶ PBMC labeled and analyzed using a Fortessa cytometer (BD Biosciences) and the Kaluza software (Beckman-Coulter).

3′ Singe-cell RNA sequencing. PBMC were loaded onto a Chromium Single Cell Chip (10X Genomics), and captured mRNAs were barcoded during cDNA synthesis using the Chromium Next GEM Single Cell 3′ GEM, Library & Gel Bead Kit v3.1 (10X Genomics). Libraries were sequenced on NovaSeq 6000 (Illumina). Raw BCL-files were demultiplexed using bcl2fastq (version 2.20.0.422 from Illumina) and read quality control performed using fastqc (version 0.11.9). Reads were pseudo-mapped to the Ensembl reference transcriptome v99 (Homo sapiens GRCh38 build with kallisto, version 0.46.2). The index was made with kb-python (version 0.24.4) wrapper of kallisto. Barcode correction using the whitelist provided by the manufacturer and gene-based reads quantification was performed with BUStools (version 0.40.0). Empty droplets were detected using the emptyDrops function from the dropletUtils package (version 1.10.3), barcodes with p-value <0.001 (Benjamini-Hochberg-corrected) were considered for analysis. The count matrix was filtered to exclude genes detected in less than five cells, cells with less than 1500 UMIs or less than 200 detected genes, as well as cells with mitochondrial transcripts proportion higher than 20%. Cell cycle scoring was performed using the CellcycleScoring function of Seurat package (version 4.0.0), and the cyclone function of Scran (version 1.18.5). Doublets were discarded using scDblFinder (version 1.4.0) and scds (version 1.6.0). We manually verified that cells identified as doublets did not correspond to cells in G2M phase. Datasets were integrated using the Harmony method, merged using Seurat (version 4.0.4), and the SCTransform normalization method was used to normalize, scale, select 3000 Highly Variable Genes and regress out bias factors. The reduced PCA spaces were used as input for the HarmonyMatrix function implemented in Harmony package (version 0.1.0) where the batch effect (orig.ident) was regressed. The shared space output by harmony was used for clustering. The optimal number of dimensions was evaluated by assessing a range of reduced Harmony spaces using 3 to 49 dimensions, with a step of 2. For space, Louvain clustering of cells was performed using a range of values for the resolution parameter from 0.1 to 1.2 with a step of 0.1. The optimal space was the combination of kept dimensions and clustering resolution resolving the best structure (clusters homogeneity and compacity) in a UMAP. Marker genes for Louvain clusters were identified through a «one versus others» differential analysis using the Wilcoxon test through the FindAllMarkers function from Seurat, considering only genes with a minimum log fold-change of 0.5 in at least 75% of cells from one of the groups compared, and FDR-adjusted p-values <0.05 (Benjaminin-Hochberg method). UMAP visualization was done using Cerebro (version 1.2.2).

Cytokine level measurements. Sorted IGRANs were cultured for 24 hours at 1 million/mL in RPMI medium. Supernatants were collected and centrifuged at 1500 rpm for 10 min and stored at −80° C. Medium without IGRAN was used as control (N=3). Plasma aliquots were centrifuged at 1500 rpm for 15 min at 4° C., diluted 1:4 and analyzed using Bio-Plex Pro™ Human Chemokine Panel 40-plex Assay (Bio-rad). Acquisitions and analyses were performed on a Bio-Plex 200 system with Manager 6.1 Software (Bio-rad), respectively. Soluble Calprotectin (1:100) and S100A12 (1:2) were measured using R-plex Human Antibody Sets (Meso Scale Discovery), a MESO QuickPlex SQ120 reader and the MSD's Discovery Workbench 4.0. Each sample assayed twice, average value taken as final result.

Lymphocyte proliferation assay. Ten million of PBMC were stained with anti-CD15, -CD16, -CD66b, -CD45 and -CD14 antibodies before sorting CD45⁺CD15⁺CD16⁻CD66b⁺CD14⁻ cells (IGRAN) using an Influx cell sorter (BD Biosciences). Total PBMC and IGRAN-depleted PBMC were suspended in Cell trace Violet (5 μM in PBS 1×, Thermo Fisher Scientific) for 15 min at 37° C., then plated in 96-well round bottom plates (1 million/mL in complete RPMI medium). When indicated, IGRANs were added to IGRAN-depleted PBMC (1:10 ratio). In cultures, T-cells were activated in wells coated with anti-CD3 (eBiosciences, clone OKT3) and anti-CD28 (eBiosciences, clone CD28.2) antibodies in IL-2-containing medium (0.01 ug/ml, Peprotech) for 4 days. Cells were labeled with LIVE/DEAD™ Fixable Blue Dead Cell Stain Kit (Thermo Fisher Scientific) and antibodies and analyzed using a Fortessa.

Reagents, CXCR1/2 inhibitors Reparixin and Ladarixin were from MedChemTronica and Clinisciences, respectively, dissolved in dimethylsulfoxide (DMSO). Stem cell factor recombinant (SCF), interleukin-3 recombinant (IL-3), thrombopoietin recombinant (TPO), Fms-like tyrosine kinase 3 recombinant (FLT-3) and Interleukin 8 (IL-8 or CXCL8) were from Peprotech.

CD34⁺ cultures, CD34⁺ cells were cultured for 72 h at 1×10⁵ cells/ml in complete MEM-alpha medium, SCF (50 ng/mL), IL-3 (10 ng/mL), TPO (10 ng/mL) and FLT-3 (50 ng/mL mL), to which CXCL8 was added in a 37° C. incubator with 5% CO₂. Cells were counted after Trypan blue staining. For methylcellulose assays, CD34+ cells were plated in duplicate, at 500 cells with 1 mL complete methylcellulose (MethoCult™ H4034 Optimum, Stem cell) with indicated doses of CXCL8. Colonies were enumerated and phenotyped at day 14.

RNA extraction, RT analysis. Total RNA was obtained from frozen dry pellet of sorted CD14⁺, CD3⁺ using Trizol™ Reagent (Thermo Fischer Scientific) and Direct-zol™ RNA Miniprep (Zymo research). For IGRAN, total RNAs were extracted using RLT buffer (Quiagen) and Trizol™ LS Reagent. Precipitated RNA was purified on a ‘¹mini-RNA’¹ column (RNeasy Mini Kit from Qiagen), quantified on Nanodrop (Spectrophotometer ND-1000) and stored at −80° C. Total RNA was reverse transcribed with SuperScript IV reverse transcriptase with random hexamers (Thermo Fisher Scientific). Real-time quantitative polymerase chain reaction (RT-qPCR) was performed with AmpliTaq Gold polymerase in an Applied Biosystems 7500 thermocycler (Thermo Fisher Scientific). Primer sequences are available upon request.

Bulk RNA sequencing. RNA integrity (RNA Integrity Score ≥7.0) was checked on the Fragment Analyzer (Agilent) and quantity was determined using Qubit (Invitrogen). SureSelect Automated Strand Specific RNA Library Preparation Kit was used with the Bravo Platform. Briefly, 100 ng of total RNA sample was used for poly-A mRNA selection using oligo(dT) beads and subjected to thermal mRNA fragmentation before conversion into double stranded DNA for library preparation. Final libraries were bar-coded, purified, pooled and paired-end sequenced on Novaseq-6000 sequencer (Illumina) at Gustave Roussy. Raw reads were mapped to hg19 genome with Tophat2 (v2.0.14)/Bowtie2 (v2.1.0). The number of reads per gene (GENECODE gene annotation v241ift37) was counted using HTSeq (0.5.4p5) and DESeq2 (v1.10.1) package was used for differential gene expression analysis. Gene Set Enrichment Analysis was performed using enrichplot package with a number of Permutation: 10 000, min Gene Set Size: 20, max Gene Set Size: 800, pvalueCutoff 0.05.

Whole Exome Sequencing (WES). DNA collected from sorted CD14⁺, CD3⁺ and IGRANs was assayed on Nanodrop, and 200 ng genomic DNA were sheared with the Covaris E220 system (LGC Genomics/Kbioscience). Fragments were end-repaired, extended with an A base at the 3′ end, ligated with paired-end adapters with the Bravo platform (Agilent) and amplified for ten cyclesFinal libraries were paired-end sequenced (2×100 bp reads) using Illumina NovaSeq-6000 sequencer. Somatic variants were detected in monocytes and IGRANs, using CD3 T-cells as a control, and validated on IGV software.

Statistical analysis. Participants' characteristics were reported as numbers and percentages for categorical variables, mean and standard deviation (normal distribution) or median and interquartile range (skewed distribution) for continuous variables. Differences between groups were compared using Mann-Whitney test (2 groups) or Kruskal-Wallis test (>2 groups). Correlation between clinical and biological parameters were assessed with the Spearman method. The p-values of the tests are expressed as follows: * p<0.05, ** p<0.01, *** p<0.001, **** p<0.0001. In the absence of precision, the test is not significant. A Cox proportional hazards model was used to adjust the effects of IGRAN fraction (%) or absolute number (×10⁹/L) as a continuous variable on overall survival (OS), defined as the time between the date of diagnosis and the date of death, whatever the cause (for patients who remain alive, OS was censored on the date of last follow-up), and event-free survival (EFS), defined as the time between the date of diagnosis and date of AML transformation or death due to any cause whichever occurs first (for patients who remain alive without AML transformation, EFS was censored on the date of last follow-up. The variables included in the Group Francophone des Myélodysplasies score (GFM) (24) were used in the multivariate models (Age, WBC>15×10⁹/L, hemoglobin <10 g/L, platelets <100×10⁹/L, ASXL1 mutations). The optimal cut-points were computed using maximally selected log-rank statistic (maxstat R package)(51,52) for overall survival to define two prognostic groups, and the Kaplan-Meyer method was used for survival curves. SAS 9.4 (SAS Institute Inc. Cary, NC) and R version 4.0.5 (R Foundation for Statistical Computing) softwares were used.

### Results

### Identification and Characterization of Immature Granulocytes

The prognostic significance of IMC detection ≥1% was first assessed on routine blood smears in a cohort of 580 consecutive CMML patients at diagnosis from Mayo Clinic (median age: 71 years [18-95]; 68% males) (Supplementary Table 1).

Supplementary Table 1

| | | CMML with | CMML w/o | |
|---|---|---|---|---|
| | All patients | IMCs | IMCs | P value |
| Number | 580 | 351 | 229 | |
| Age in years; median (range) | 71 (18-95) | 70 (18-95) | 72 (20-95) | 0.06 |
| Males; n (%) | 392 (68) | 235 (67) | 157 (69) | 0.68 |
| Hemoglobin, g/dl; median | 10.7 (4.3-17) | 10.5 (4.3-16.9) | 11.3 (6.4-17) | 0.005 |
| (range) | | | | |
| WBC × 10⁹/L; median (range) | 12.9 (1.3-264.8) | 18.4 (1.8-185.7) | 7.9 (1.3-264.8) | <0.0001 |
| ANC × 10⁹/L; median (range) | 6.2 (0-151) | 8.9 (0.06-151) | 3.8 (0-142.9) | <0.0001 |
| AMC × 10⁹/L; median (range) | 2.7 (0-84) | 3.3 (0.1-84) | 1.7 (0-24.6) | <0.0001 |
| ALC × 10⁹/L; median (range) | 1.9 (0-22) | 2.15 (0-14.5) | 1.6 (0.2-22) | <0.0001 |
| Platelets × 10⁹/L; median (range) | 101 (7-1473) | 97 (7-1473) | 109 (12-1277) | 0.17 |
| PB blast %; median (range) | 0 (0-19) | 0 (0-19) | 0 (0-8) | <0.0001 |
| BM blast %; median (range) | 3 (0-19) | 3 (0-19) | 2 (0-19) | 0.04 |
| Abnormal cytogenetics; n (%) | 178/551 (32) | 114/330 (35) | 64/221 (29) | 0.16 |
| Proliferative: n (%) | 288 (50) | 231 (66) | 57 (25) | <0.0001 |
| Dysplastic; n (%) | 292 (50) | 120 (34) | 172 (75) | |
| CMML-0; n (%) | 328 (57) | 172 (49) | 156 (68) | <0.0001 |
| CMML-1; n (%) | 147 (25) | 103 (29) | 44 (19) | |
| CMML-2; n (%) | 104 (18) | 75 (21) | 29 (13) | |
| Next generation | 342 | 201 | 141 | |
| sequencing analysis; n | 180 (53) | 104 (52) | 76 (55) | 0.59 |
| TET2 | 16 (5) | 9 (4) | 7 (5) | 0.83 |
| DNMT3A | 6 (2) | 5 (3) | 1 (1) | 0.19 |
| IDH1 | 20 (6) | 12 (6) | 8 (6) | 0.9 |
| IDH2 | 200 (54) | 130 (59) | 70 (46) | 0.01 |
| ASXL1 | 13 (4) | 10 (5) | 3 (2) | 0.16 |
| EZH2 | 33 (10) | 17 (8) | 16 (11) | 0.37 |
| RUNX1 | 3 (1) | 2 (1) | 1 (1) | 0.77 |
| BCOR | 17 (5) | 7 (3) | 10 (7) | 0.13 |
| SF3B1 | 158 (46) | 99 (49) | 59 (42) | 0.19 |
| SRSF2 | 25 (7) | 11 (5) | 14 (10) | 0.12 |
| U2AF1 | 11 (3) | 7 (3) | 4 (3) | 0.73 |
| ZRSR2 | 30 (9) | 17 (8) | 13 (9) | 0.8 |
| JAK2 V617F | 1 (0.3) | 1 (0.5) | 0 (0) | 0.3 |
| MPL | 50 (15) | 33 (16) | 17 (12) | 0.25 |
| CBL | 19 (6) | 10 (5) | 9 (6) | 0.57 |
| KRAS | 51 (15) | 32 (16) | 19 (13) | 0.53 |
| NRAS | 10 (3) | 7 (3) | 3 (2) | 0.45 |
| PTPN11 | 3 (1) | 3 (1.5) | 0 (0) | 0.07 |
| CSF3R | 11 (3) | 4 (2) | 7 (5) | 0.12 |
| C-KIT | 9 (3) | 6 (3) | 3 (2) | 0.62 |
| FLT3TKD | 1 (0.3) | 0 (0) | 1 (1) | 0.18 |
| NPM1 | 10 (3) | 7 (3) | 3 (2) | 0.45 |
| Tp53 | 40 (12) | 25 (12) | 15 (11) | 0.6 |
| SETBP1 | | | | |
| Leukemic transformation; n (%) | 116 (20) | 73 (21) | 43 (19) | 0.55 |
| Deaths: n (%) | 398 (69) | 245 (70) | 153 (67) | 0.44 |

Clinical and laboratory characteristics of CMML patients stratified by the presence or absence of circulating immature myeloid cells (IMCs) in the peripheral blood at diagnosis; continuous variables, median (range); dichotomized variables (n, %). ALC: absolute lymphocyte count; AMC: absolute monocyte count; ANC: absolute neutrophil count; WBC: white blood cell count; PB: peripheral blood; BM: bone marrow. Mann-Whitney test or Kruskal-Wallis test. The bold values represent p values that are statistically significant; p<0.05.

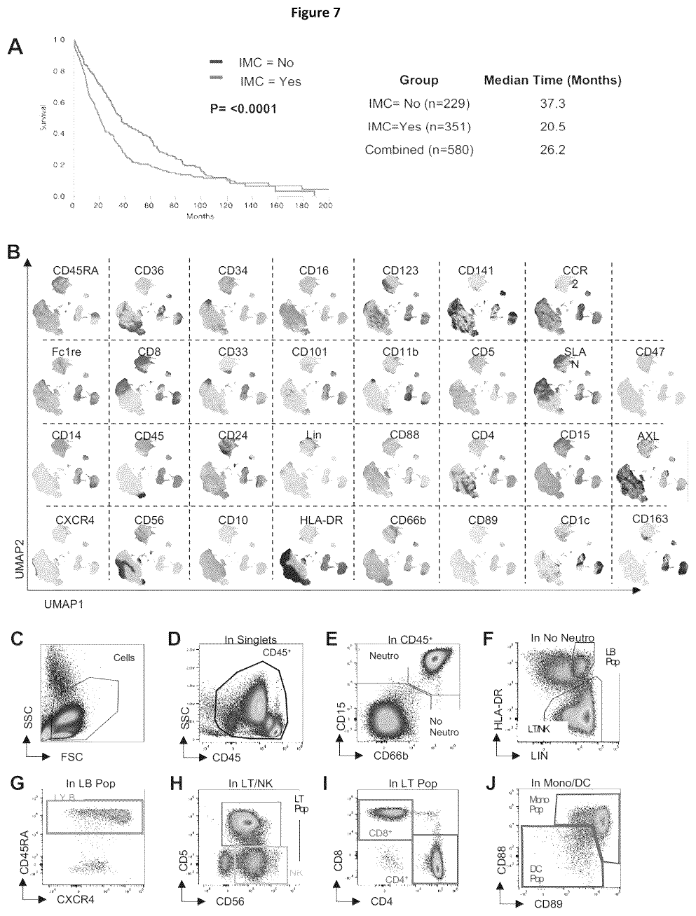

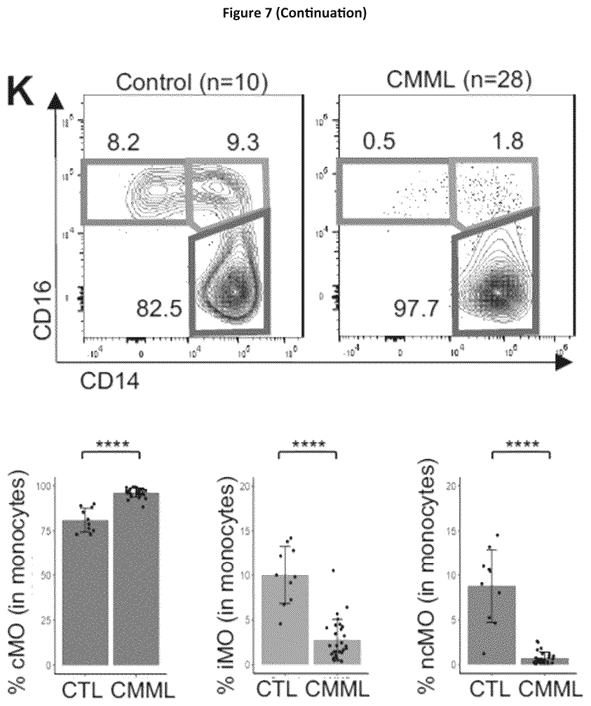

The overall survival (OS) of the 351 patients with IMC was significantly lower than that of the 229 patients without IMC, suggesting a poor prognostic factor (FIG. 7A). However, IMC is not part of most scoring systems proposed in CMML, which may be related to the limited reproducibility of IMC quantification on routine complete blood counts (19). It was explored whether flow cytometry phenotyping of peripheral blood cells could refine IMC identification and characterization.

Spectral flow cytometry was used with a panel of 34 antibodies recognizing cell surface markers (Supplementary Table 2) to generate an overview of cell populations in the peripheral blood.

TABLE 2
Supplementary. List and references of antibodies used in indicated assays.

| | | | | |
|---|---|---|---|---|
| | | CAT, | | |
| REAGENT | COLOR | NUMBER | CLONE | SOURCE |

Antibodies for Spectral flow cytometry

| | | | | |
|---|---|---|---|---|
| CD24 | BB700 | 566525 | ML5 | BD Biosciences |
| CD1c | BUV563 | 748724 | F10/21A3 | BD Biosciences |
| CD141 | BV421 | 565321 | 1A4 | BD Biosciences |
| CD101 | BV605 | 747548 | V7.1 | BD Biosciences |
| CD163 | BUV737 | 741863 | GHI/61 | BD Biosciences |
| CD34 | BUV661 | 749902 | 581 | BD Biosciences |
| CD36 | BUV496 | 750114 | CLB-IVC7 | BD Biosciences |
| CD5 | BV750 | 747108 | UCHT2 | BD Biosciences |
| CD45RA | BUV395 | 740315 | 5H9 | BD Biosciences |
| CD15 | PE-Dazzle 594 | 562463 | HI98 | BD Biosciences |
| CD16 | BUV805 | 748850 | 3G8 | BD Biosciences |
| FcER1a | eFluor 450 | 48-5899-41 | AER-37 (CRA1) | Thermofisher |
| NKG2D | PerCP-eFluor 710 | 46-5878-41 | 1D11 | Thermofisher |
| CD3 | PerCP-eFluor 710 | 46-0037-41 | OKT3 | Thermofisher |
| CD7 | PerCP-eFluor 710 | 46-0078-41 | 4H9 | Thermofisher |
| CD19 | PerCP-eFluor 710 | 46-0198-42 | SJ25C1 | Thermofisher |
| CD123 | superbright436 | 62-1239-42 | 6H6 | Thermofisher |
| SLAN | FITC | 130-117-371 | DD1 | Miltenyibiotech |
| AXL | APC | FAB154A | #108724 | R&D |
| CD66b | Alexa Fluor 647 | 305109 | G10F5 | Biolegend |
| CD89 | APC-Fire 750 | 354115 | A59 | Biolegend |
| CCR2 | BV785 | 357233 | K036C2 | Biolegend |
| CD11b | BV650 | 101239 | M1/70 | Biolegend |
| CD33 | BV570 | 303417 | WM53 | Biolegend |
| HLA-DR | PE-Fire810 | | | Biolegend |
| CD14 | Sparkle blue 550 | 367147 | 63D3 | Biolegend |
| CD56 | PE-Fire 700 | 392427 | NCAM | Biolegend |
| CXCR4 | PE-CY5 | 306508 | 12G5 | Biolegend |
| CD45 | Percp | 304026 | HI30 | Biolegend |
| CD10 | PE-CY7 | 312214 | HI10A | Biolegend |
| CD8 | BV510 | 344732 | SK1 | Biolegend |
| CD88 | PE | 344303 | C5aR | Biolegend |
| CD47 | BV711 | 563761 | B6H12 | BD Biosciences |
| CD4 | CfluorYG584 | | SK3 | CYTEK |

Antibodies for conventionnal flow cytometry

| | | | | |
|---|---|---|---|---|
| CD24 | BUV395 | 563818 | ML5 | BD Biosciences |
| CD15 | PE-Dazzle 594 | 562463 | HI98 | BD Biosciences |
| CD16 | APC-H7 | 560195 | 3G8 | BD Biosciences |
| CD3 | APC | 555342 | HIT3a | BD Biosciences |
| CD19 | APC | 55415 | SJ25C1 | BD Biosciences |
| CD66b | FITC | 555724 | G10F5 | BD Biosciences |
| CD11b | Percp-CY5.5 | 301328 | ICRF44 | Biolegend |
| CD33 | PE | 555450 | WM53 | BD Biosciences |
| HLA-DR | BV421 | 562804 | G46-6 | Biolegend |
| CD14 | PE-CY7 | A22331 | RMO52 | Beckman Coulter |
| CD56 | APC | IM2474 | N901 | Beckman Coulter |
| CD45 | BV510 | 304036 | HI30 | Biolegend |

Antibodies for semi-solid culture analysis

| | | | | |
|---|---|---|---|---|
| CD163 | BUV737 | | | |
| CD15 | PE-Dazzle 594 | 562463 | H198 | BD Biosciences |
| CD66b | Alexa Fluor 647 | 305109 | G10F5 | Biolegend |
| CD11b | BUV805 | | | Biolegend |
| CD45 | BV510 | 304036 | HI30 | Biolegend |
| CD14 | Sparkle blue 550 | 367147 | 63D3 | Biolegend |
| CD36 | BUV496 | 750114 | CLB-IVC7 | BD Biosciences |
| GPA | PE | | | |

Antibodies for proliferation assays

| | | | |
|---|---|---|---|
| CD3 | BUV738 | UCHT1 | BD Biosciences |
| CD4 | APC | OKT4 | Biolegend |
| CD8 | BV786 | RPA-T8 | BD Biosciences |

Antibodies for bone marrow CD34+ analysis

| | | | | |
|---|---|---|---|---|
| CD45 | APC-H7 | 561863 | HI30 | BD Biosciences |
| CD34 | PE-CY7 | 560710 | | BD Biosciences |
| CD38 | eFluor 450 | 48-0388-42 | | Thermofisher |
| CXCR1 CD181 | BV650 | 749071 | | BD Biosciences |
| CXCR2 CD182 | BUV737 | 743422 | | BD Biosciences |

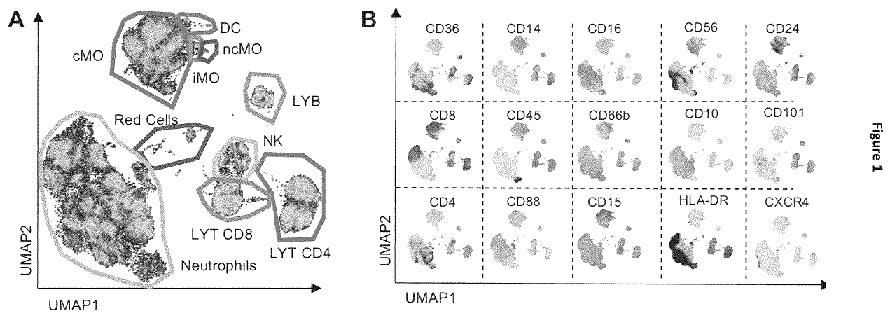

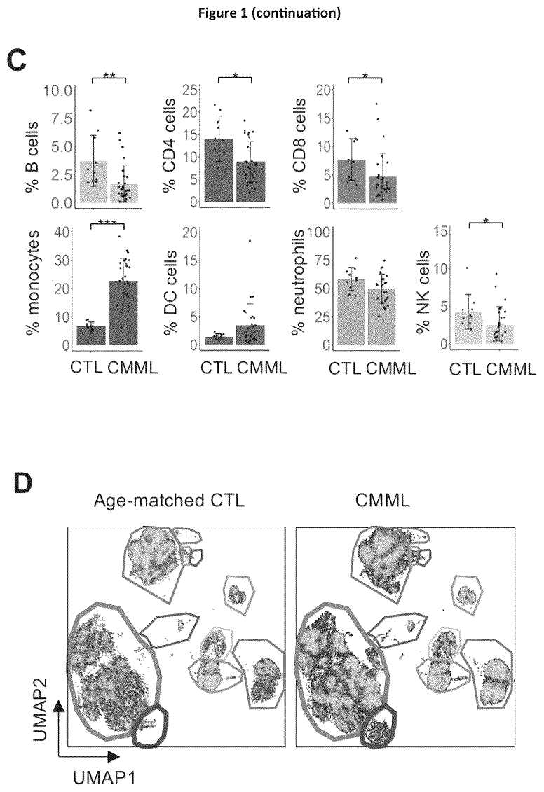

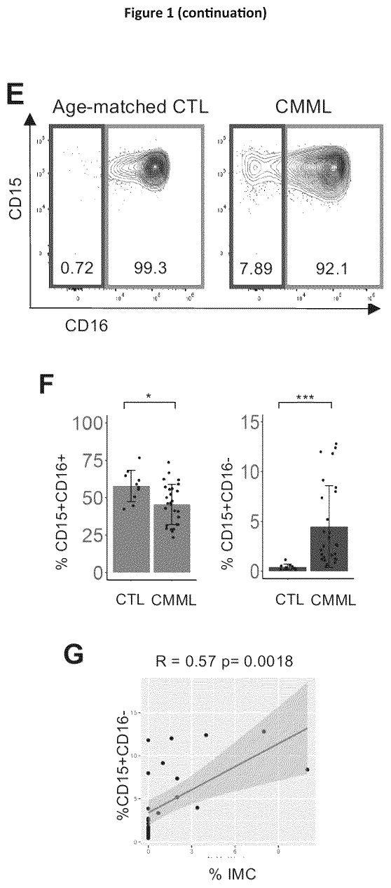

Pooled data from untreated CMML patients (n=27) and age-matched controls (n=10) (Supplementary Table 3) were subjected to a clustering analysis and visualized after a dimensionality reduction using the unsupervised uniform manifold approximation and projection (UMAP) algorithm (20) (FIG. 1A, FIG. 7B).

| | Learning cohort: spectral flow cytometry | | | | | Validation cohort: conventionnal flow cytometry | | | | |
|---|---|---|---|---|---|---|---|---|---|---|
| | Aged-controls | | CMML | | p value | Aged-controls | | CMML | | p value |
| Number | 10 | | 27 | | | 64 | | 209 | | |
| Age in years | 71.5 | (65-79) | 76.0 | (60-92) | 0.17 | 74.0 | (65-94) | 75.0 | (50-93) | 0.95 |
| Males; n (%) | 4 | (40%) | 16 | (60%) | | 28 | (46%) | 132 | (63%) | |
| Hemoglobin, g/dL | 13.9 | (13.1-15.8) | 12.4 | (8.4-16.2) | 0.01 | 13.4 | (10.7-16.8) | 11.9 | (6.4-17.2) | <0.0001 |
| WBC × 10⁹/L | 5.5 | (4.4-7.7) | 7.2 | (3.1-19.9) | 0.085 | 6.9 | (4.5-12.5) | 10.7 | (2.7-141.9) | <0.0001 |
| ANC × 10⁹/L | 3.1 | (2.3-5.8) | 3.2 | (1.2-9.4) | 0.64 | 4.3 | (1.9-7.0) | 5.4 | (0.5-65.9) | 0.0042 |
| Neutrophils (%) | 57.3 | (40.0-75.3) | 50.3 | (29.9-77.0) | 0.129 | 61.5 | (36.0-78.0) | 51.0 | (7.0-93.8) | <0.0001 |
| AMC × 10⁹/L | 0.4 | (0.3-0.6) | 1.65 | (1-6.6) | <0.0001 | 0.6 | (0.3-0.9) | 2.22 | (1-59.7) | <0.0001 |
| Monocytes (%) | 7.5 | (6.0-11.0) | 28 | (12.8-42.0) | <0.0001 | 8.7 | (3.7-17.8) | 23.0 | (10.0-64.0) | <0.0001 |
| ALC × 10⁹/L | 1.5 | (1.1-3.1) | 1.4 | (0.4-3.8) | 0.27 | 1.9 | (1.2-2.9) | 1.9 | (0.3-10) | 0.6 |
| Lymphocytes (%) | 29.0 | (15.6-43.8) | 22.2 | (3.1-39.5) | 0.006 | 27 | (16-51) | 18 | (1.4-50) | <0.0001 |
| Platelets × 10⁹/L | 227 | (183-369) | 127 | (25-1097) | 0.0009 | 234 | (152-431) | 111 | (12-688) | <0.0001 |

| Abnormal karyotype/ | NA | 2/18 | (11%) | NA | 46/190 | (24%) |
|---|---|---|---|---|---|---|
| tested; n (%) | | | | | | |
| Proliferative; n (%) | NA | 23 | (85%) | NA | 83 | (40%) |
| Dysplastic; n (%) | NA | 4 | (15%) | NA | 126 | (60%) |
| CMML-0; n (%) | NA | 15 | (55%) | NA | 104 | (49%) |
| CMML-1; n (%) | NA | 10 | (38%) | NA | 77 | (36.5%) |
| CMML-2; n (%) | NA | 2 | (7%) | NA | 31 | (14.5%) |
| Classical monocytes | NA | 97.5% | (94.4-99.5) | NA | 96.9% | (69-99.5) |
| (% total monocytes) | | | | | | |
| GFM prognosis | | 27 CMML | | | 180 CMML | |
| model, N | | tested | | | tested | |
| GFM, low; n (%) | NA | 21 | (78%) | NA | 93 | (52%) |
| GFM, intermed; n (%) | NA | 4 | (15%) | NA | 59 | (33%) |
| GFM, high; n (%) | NA | 2 | (7%) | NA | 28 | (15%) |
| Next generation | | 27 CMML | | | 180 CMML | |
| sequencing | | tested | | | tested | |
| analysis; n | | | | | | |
| TET2 mutated n (%) | NA | 23 | (85%) | NA | 120 | (67%) |
| SRSF2 mutated n (%) | NA | 15 | (55%) | NA | 64 | (36%) |
| ASXL1 mutated n (%) | NA | 11 | (41%) | NA | 48 | (27%) |
| NRAS mutated n (%) | NA | 2 | (7%) | NA | 22 | (12%) |
| KRAS mutated n (%) | NA | 3 | (11%) | NA | 21 | (12%) |
| CBL mutated n (%) | NA | 5 | (19%) | NA | 14 | (8%) |
| AML1 mutated n (%) | NA | 2 | (7%) | NA | 33 | (18%) |

Supplementary Table 3. Characteristics of CMML patients and age-matched controls tested by spectral flowcytometry (learning cohort, left columns) or by conventional flow cytometry (validation cohort, right columns). Continuous variables, median (range); dichotomized variables (n, %).); dichotomized variables (n, %). ALC: absolute lymphocyte count; AMC: absolute monocyte count; ANC: absolute neutrophil count; WBC: white bloodcell count; GFM: groupe francophone des myelodysplasies. Mann-Whitney test. The bold values represent p values that are statistically significant; p<0.05. No information available for young donors

This approach allowed identifying classical, intermediate and nonclassical monocytes; dendritic cells; B cells; CD4⁺ and CD8⁺ T cells; natural killer (NK) cells; and neutrophils (18), A gating strategy was applied to precisely quantify each cell population in every sample among white blood cells (CD45⁺ cells, FIGS. 7 C-J). Examination of the cell-type profiles of samples from patients with CMML and controls revealed the increased fraction of circulating monocytes that defines CMML (FIG. 1C), with a typical accumulation of classical monocytes at the expense of intermediate and nonclassical monocytes (FIG. 7K). The fractions of circulating B cells, CD4⁺ and CD8⁺ T cells and NK cells decreased in CMML patients compared to age-matched controls, whereas no significant changes were observed in the global dendritic cell and granulocyte populations (FIG. 1C).

A detailed analysis of neutrophils detected a cluster of cells in CMML samples that was hardly observed in age-matched controls (FIG. 1D. This cell subset expressed high levels of CD15, CD24 and CXCR4; low levels of CD45, CD10, and CD101; and almost no CD16, suggestive of immature granulocytes (IGRANs) (FIG. 1). Quantification of the neutrophil populations among CD45⁺ cells in individual samples revealed a significant decrease in the fraction of mature CD15⁺CD16⁺ neutrophils in the CMML group compared to the healthy donor group, while the fraction of CD15⁺CD16⁻ cells significantly increased with high interpatient heterogeneity (FIG. 1E, 1F). The fraction of CD15⁺CD16⁻ neutrophils correlated with the IMC fraction detected on routine blood smears (r=0.57, FIG. 1G). Altogether, spectral flow cytometry analysis of CMML peripheral blood cells refined the detection of IMC and characterized a subset of cells as CD15⁺CD16⁻CD66b⁺ IGRANs.

### Flow Cytometric Detection of IGRANs Refines CMML Prognostic

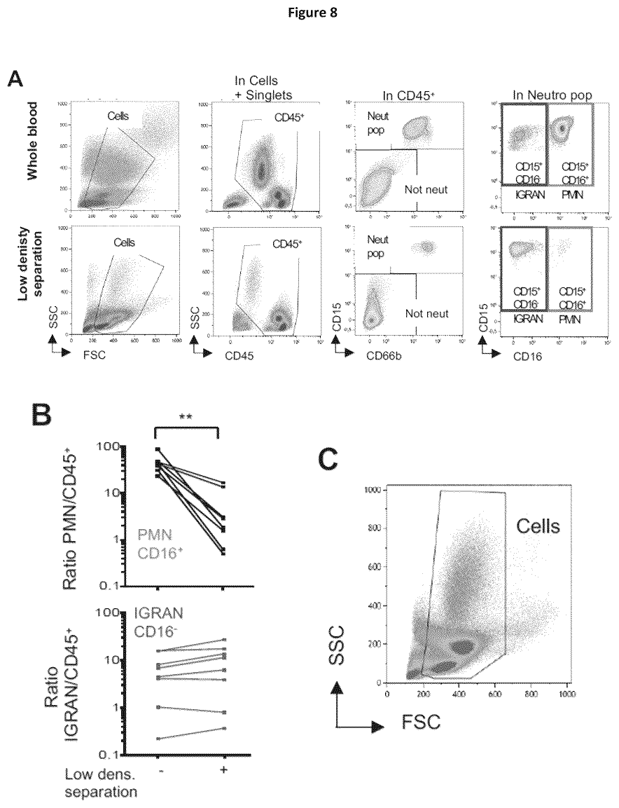

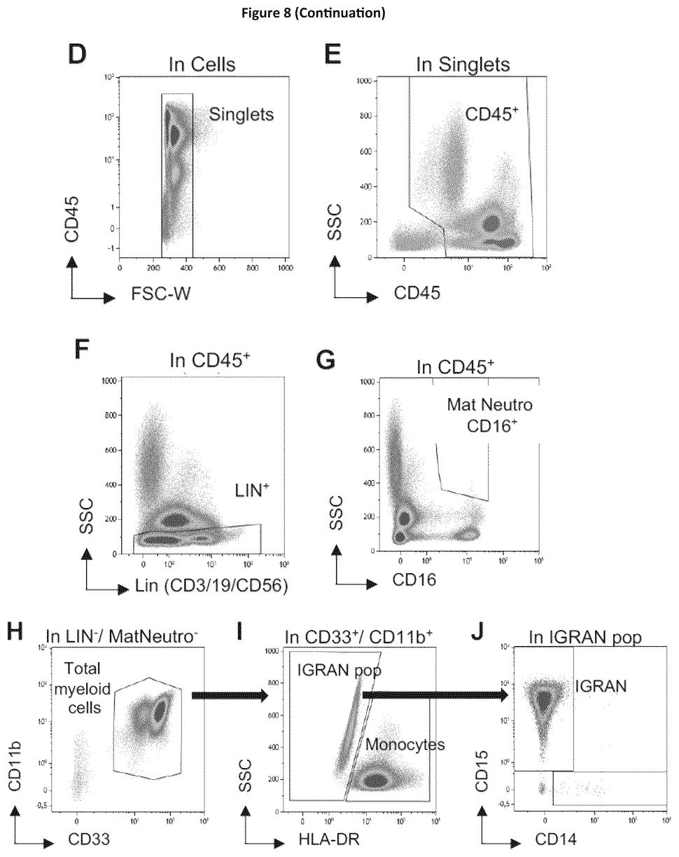

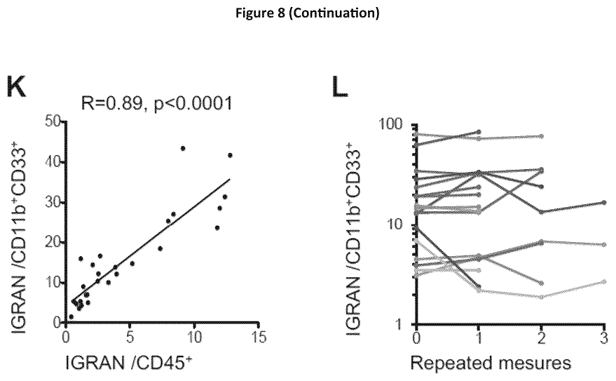

Having identified informative phenotypic markers by spectral flow cytometry analysis, a conventional, multiparametric flow cytometry assay was developed (Supplementary Table 2) to routinely quantify the fraction of IGRANs in freshly collected blood samples and revisit the prognostic significance of this parameter in CMML. It was noticed that IGRANs were part of the peripheral blood mononucleated cell (PBMC) population sorted by low density gradient centrifugation, a process that removes a majority of CD15⁺CD16⁺CD66b⁺ mature neutrophils without affecting IGRAN population (FIG. 8A, 8B). Based on this low-density cell property, the conventional flow cytometry assay was performed on PBMC (FIGS. 8 C-J). The IGRAN fraction measured among Lin⁻CD16⁻CD11b⁺CD33⁺ cells correlated with that among total CD45⁺ cells (Fire 8K) With this conventional flow cytometry assay, repeated measures of the IGRAN fraction over up to one year in 17 untreated CMML patients showed reproducible results (FIG. 8L).

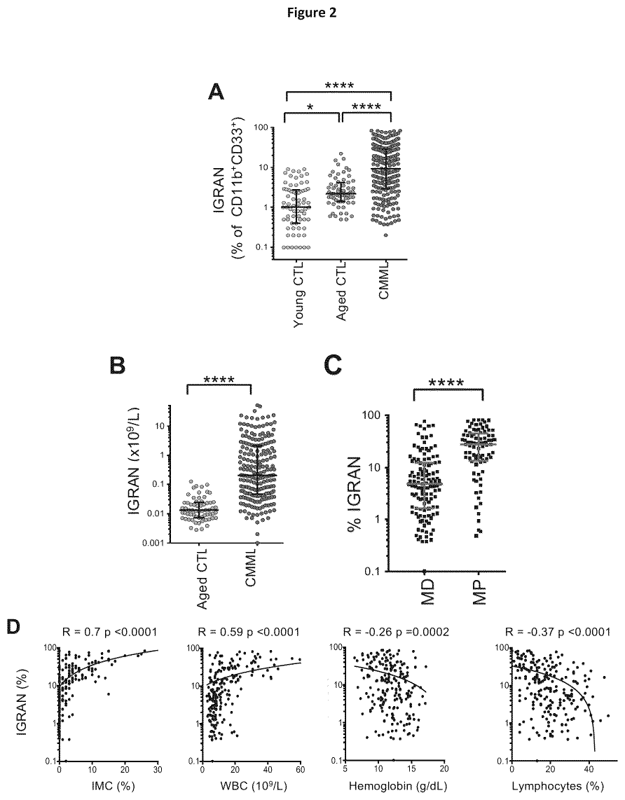

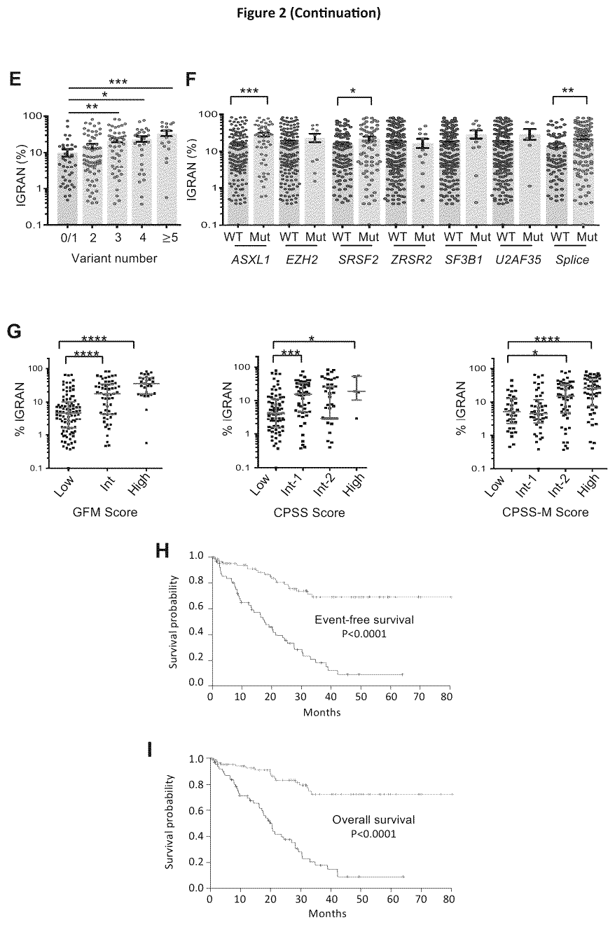

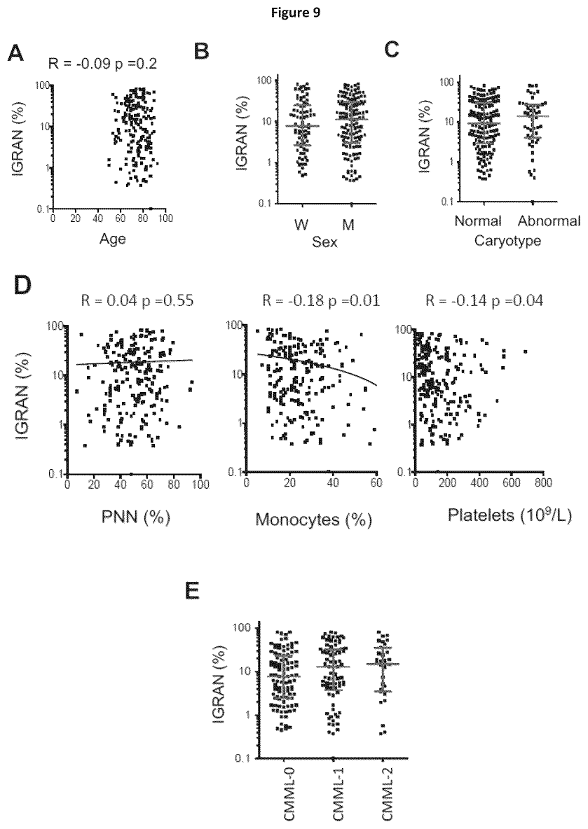

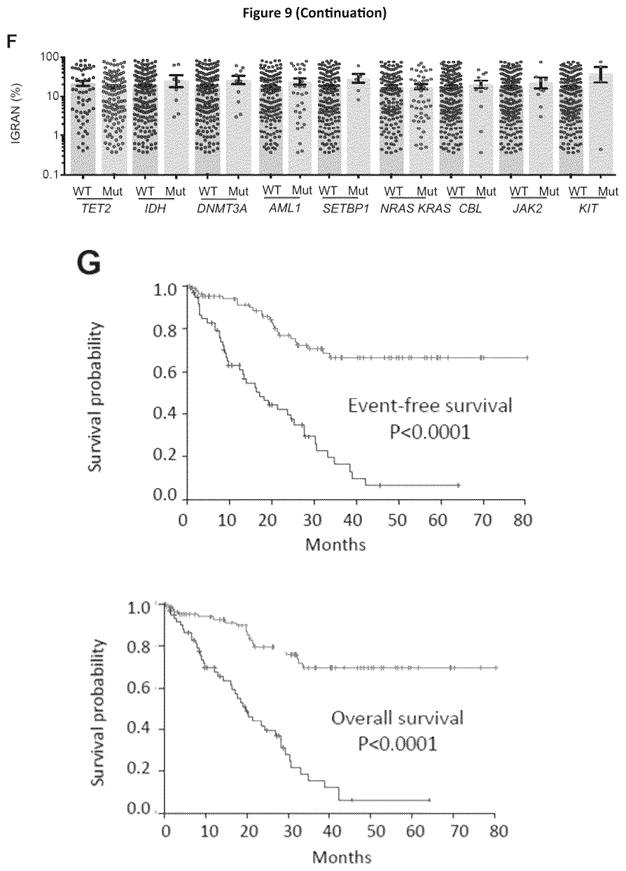

This flow cytometry assay was prospectively assessed between March 2015 and April 2019 on a validation cohort of 209 untreated CMML patients (median age: 75 years [50-93], 63% males) compared to 64 age-matched controls (median age: 74 years [65-94]) and 71 younger healthy donors (<65 years old) (Supplementary Table 3). CMML diagnosis was supported by flow cytometry analysis of peripheral monocyte subsets (21): in 192 patients (92%), classical monocytes represented more than 94% of total monocytes, while a decreased fraction of slan⁺ nonclassical monocytes was observed in 15 of the 17 remaining patients (7%)(22,23). The fraction of IGRANs among myeloid cells was significantly higher in aged controls (median 2.2% [0.5-22%]) than in younger ones (median 1.0% [0-0.9%], p=0.0001) and was further increased in CMML patients (median 9.2% [0.03-82.5%], p<0.0001) (FIG. 2A). The absolute number of circulating IGRANs also was statistically increased in CMML patients, sometimes reaching values close to 50×10⁹/L, representing a 1000-fold increase over the values in aged controls (FIG. 2B). The fraction of IGRANs was significantly higher in MP-CMML than in MD-CMML (FIG. 2C) and positively correlated with the fraction of IMCs quantified on blood smears (r=0.7, p<0.0001). However, the flow cytometric quantification of IGRANs has a more precise detection threshold for patients with low IMC (FIG. 2D). The fraction of IGRANs also correlated with WBC count and with lower hemoglobin level and lymphocyte fraction (FIG. 2D). In contrast, no correlation was observed for IGRAN fraction with age, sex, presence of cytogenetic abnormalities or total neutrophil fraction, and the correlations with monocyte fraction, platelet count and WHO-defined subtypes remained weak (FIG. 9 A-E). Analysis of mutations in a panel of 35 genes recurrently mutated in CMML (Supplementary Table 3) showed an increase in the IGRAN fraction with an increasing number of mutated genes as well as with the presence of ASXL1 or SRSF2 gene mutations (FIG. 2E-F). No significant association was detected with the other common variants in CMML, including those in signaling genes (FIG. 9 F). Finally, the IGRAN fraction increased with disease risk level, as measured using the Groupe Francophone des Myelodysplasies score (GFM)(24), the CMML-specific prognostic scoring system (CPSS)(25) and the CPSS-M score that incorporates molecular genetic data (26)(FIG. 2G). Together, these results indicate that IGRAN accumulation in blood is associated to poor prognosis scores and to poor prognosis markers like ASXL1 mutations, or proliferative forms of the disease. The relationship between the IGRAN fraction and other parameters were validated by analyses performed using the absolute number of circulating cells (10⁹/L) (Supplementary Table 4), indicating the robustness of IGRAN quantification to separate the most severe forms of the disease.

Supplementary Table 4.
Correlation between IGRANs (×10⁹L) and clinical and biological
disease parameters. Mann-Whitney test or Kruskal-Wallis test.

| Parameters | Number | Median [95% CI] | Significance |
|---|---|---|---|
| A-Categorical | | | |
| variables | | | |
| Sex | | | NS |
| Females | 77 | 0.2 [0.05-2.06] | |
| Males | 132 | 0.22 [0.04-2.05] | |
| WHO CMML | | | 0.019 |
| subtype 2016 | | | |
| CMML-0 | 101 | 0.15 [0.04-0.76] | |
| CMML-1 | 77 | 0.28 [0.06-2.58] | |
| CMML-2 | 31 | 0.91 [0.07-5.79] | |
| FAB CMML | | | P < 0001 |
| subtype | | | |
| Dysplastic | 126 | 0.07 [0.03-0.21] | |
| Proliferative | 83 | 2.23 [0.62-7.54] | |
| ASXL1 mutation | | | P < 0001 |
| Absent | 150 | 0.13 [0.04-0.71] | |
| Present | 53 | 2.49 [0.28-5.64] | |
| TET2 mutation | | | NS |
| Absent | 64 | 0.18 [0.04-3,42] | |
| Present | 140 | 0.21 [0.05-1.45] | |
| SRSF2 mutation | | | NS |
| Absent | 121 | 0.17 [0.04-1.07] | |
| Present | 73 | 0.36 [0.07-2.88] | |
| AML 1 mutation | | | NS |
| Absent | 168 | 0.19 [0.04-1.75] | |
| Present | 35 | 0.38 [0.07-5.01] | |
| NRAS mutation | | | 0.005 |
| Absent | 179 | 0.17 [0.04-1.51] | |
| Present | 24 | 1.31 [0.25-5.57] | |
| KRAS mutation | | | NS |
| Absent | 177 | 0.23 [0.05-2.15] | |
| Present | 26 | 0.07 [0.03-0.58] | |
| CBL mutation | | | NS |
| Absent | 187 | 0.19 [0.04-1.72] | |
| Present | 16 | 1.52 [0.1-4.65] | |
| GFM prognostic | | | P < 0001 |
| score | | | |
| Low | 116 | 0.07 [0.03-0.21] | |
| Int | 62 | 1.12 [0.21-3.03] | |
| High | 30 | 5.32 [2.35-12.06] | |
| CPSS risk | | | P < 0001 |
| stratification | | | |
| Low | 75 | 0.07 [0.03-0.19] | |
| Int-1 | 55 | 0.58 [0.10-2.15] | |
| Int-2 | 56 | 1.19 [0.24-9.45 | |
| High | S | 2.19 [1.12-5.79) | |
| CPSS-M risk | | | P < 0001 |
| stratification | | | |
| Low | 34 | 0.07 [0.03-0.19] | |
| Int-1 | 46 | 0.08 [0.04-0.25] | |
| Int-2 | 51 | 0.61 [0.1-2.75] | |
| High | 49 | 2.49 [0.34-7.55] | |
| B-Continuous variables | Number | Spearman correlation | Significance |
| Age | 209 | −0.05 | NS |
| Hemoglobin, g/dL | 209 | −0.3 | P<0001 |
| Platelets, ×10⁹/L | 209 | −0.15 | 0.02 |
| WBC count, ×10⁹/L | 209 | 0.71 | P<0001 |
| Neutrophils (%) | 209 | −0.0093 | NS |
| Monocytes (%) | 209 | −0.03 | NS |
| Lymphocytes (%) | 208 | −0.47 | P<0001 |
| IMC (%) | 187 | 0.73 | P<0001 |

Among the 209 patients included in the validation cohort, IGRAN quantification had been performed for 154 patients at diagnosis, i.e., 6 months from initial bone marrow examination. With a median follow-up of 34 months, 27 patients among 154 progressed to AML, and 62 died. The median OS and median event-free survival (EFS, defined as the time between diagnosis and AML transformation, death, or last follow-up) were 20.6 and 20 months, respectively. Univariate and multivariate Cox models were built with continuous IGRAN percentages and absolute numbers. The multivariate analysis model included clinical and mutation variables with independent prognostic value based on a previously validated scoring system (24). Both univariate and multivariate models showed a significant impact of IGRANs measured at diagnosis on EFS or OS (Supplementary Table 5).

Supplementary Table 5.
Correlation between IGRANs (expressed in fraction or absolute number × 10⁹/L and
overall survival or event-free survival (EFS, defined as the time between diagnosis and
AML transformation, death, or last follow-up) using Cox models, Multivariate model
were built after adjusting for potential confounders (Age, WBC > 15 × 10⁹/L,
hemoglobin < 10 g/L, platelets < 100 × 10⁹/L, ASKL1 mutations). The proportional hazard
hypothesis was verified by visual display of the cumulative sums of martingale residuals.

| | Hazard ratio | | Hazard ratio | |
|---|---|---|---|---|
| | [95% CI] | P- | [95% CI] | P- |
| | (univariate model) | value | (multivariate model) | value |
| Analysis with continuous | | | | |
| data | | | | |
| Overall survival (OS) | | | | |
| IGRAN (%) | 1.029 [1.019-1.038] | <0.0001 | 1.014 [1.00-1.026] | 0.0115 |
| IGRAN (10⁹/L) | 1.104 [1.074-1.135] | <0.0001 | 1.059 [1.02-1.099] | 0.0028 |
| Event-free survival (EFS) | | | | |
| IGRAN (%) | 1.028 [1.019-1.037] | <0.0001 | 1.016 [1.00-1.026] | 0.0049 |
| IGRAN (10⁹/L) | 1.103 [1.074-1,132] | <0.0001 | 1.067 [1.03-1.105] | 0.0003 |
| Analysis with an IGRAN | | | | |
| cutpoint | | | | |
| Overall survival (OS) | | | | |
| IGRAN (14%) | 5.93 [3.364-10.454] | <0.0001 | 3.678 [1.850-7.305] | 0.0002 |
| IGRAN (0.4 × 10⁹/L) | 5.49 [3.183-9.470] | <0.0001 | 3.115 [1.482-6.548] | 0.0027 |
| Event-free survival (EFS) | | | | |
| IGRAN (14%) | 5.10 [2.993-8,684] | <0.0001 | 3.003 [1.592-5.663] | 0.0007 |
| IGRAN (0.4 × 10⁹/L) | 4.79 [2.857-8.032] | <0.0001 | 2.579 [1.292-5.149] | 0.0003 |

The data were then computed to dichotomize continuous variables by selecting cutoff values to maximize the log-rank statistics and picked out an IGRAN fraction >14% of circulating myeloid cells or an absolute IGRAN number >0.4×10⁹/L as optimal values to identify the impact of IGRANs on EFS and OS (FIG. 2H-I; FIG. 9G). With these cutoff values, the IGRAN fraction improved the accuracy of existing prognostic scores, after adjusting for potential confounders (hazard ratio for IGRAN fraction in multivariate model: 3.678, 95% Cl: [1.850-7.305] for OS and 3.003, 95% Cl: [1.592-5.663] for EFS) (Supplementary Table 5). Together, flow cytometric detection of an excess of IGRAN, using an optimal threshold of 14% of circulating myeloid cells or an absolute number >0.4×10⁹/L of IGRAN, is an independent biomarker of CMML severity.

The accumulation of IGRANs in the peripheral blood of a fraction of CMML patients questions about the mechanisms involved. The accumulation of IGRANs does not reflect subclonal genetic evolution as these clonal cells express all the genetic variants identified in monocytes without detecting additional genomic event. The recently identified role of the chromatin regulator Additional sex combs-like 1 (ASXL1) in neutrophil development, based on the neutrophilic dysplasia observed in an Asx/1-truncated zebrafish model (33) and the altered transcription program depicted in Asx/1-mutated mouse granulocyte progenitors (34) may account for the correlation between IGRAN excess and ASXL1 gene mutation in CMML patients. Some other disease features may contribute to the immunosuppressive phenotype of IGRANs. For example, the proliferative CMML subtype involves myeloid progenitor hypersensitivity to GM-CSF (9), a cytokine that promotes the generation of G-MDSCs in various other settings (32). Mutations in splicing regulator genes, that also correlate with IGRAN excess, could promotes the immunosuppressive activity of G-MDSCs through activating the NF-κB signaling pathway (35,36). Finally, IGRAN excess associates with anemia and lymphocytopenia, which may be related to the immunosuppressive potential of IGRANs (27) and their pro-inflammatory phenotype (37).

### IGRANs are Myeloid-Derived Suppressive Cells

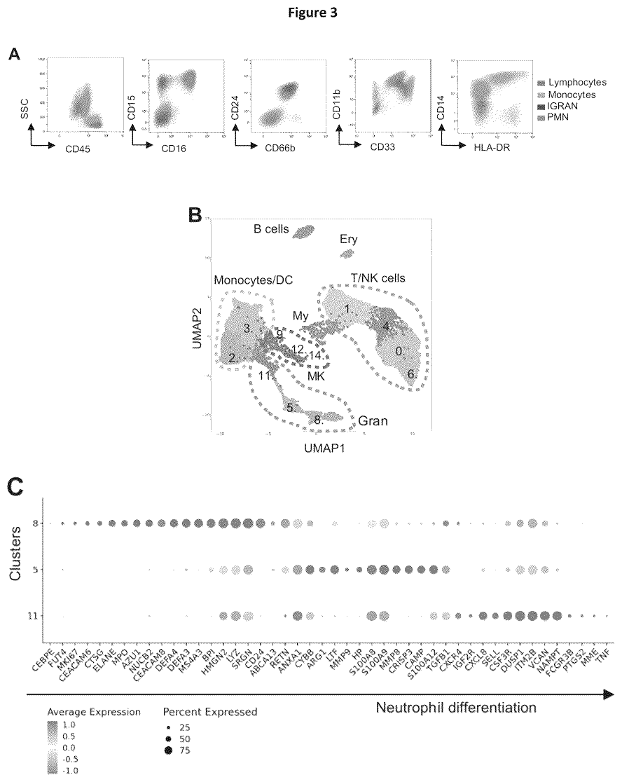

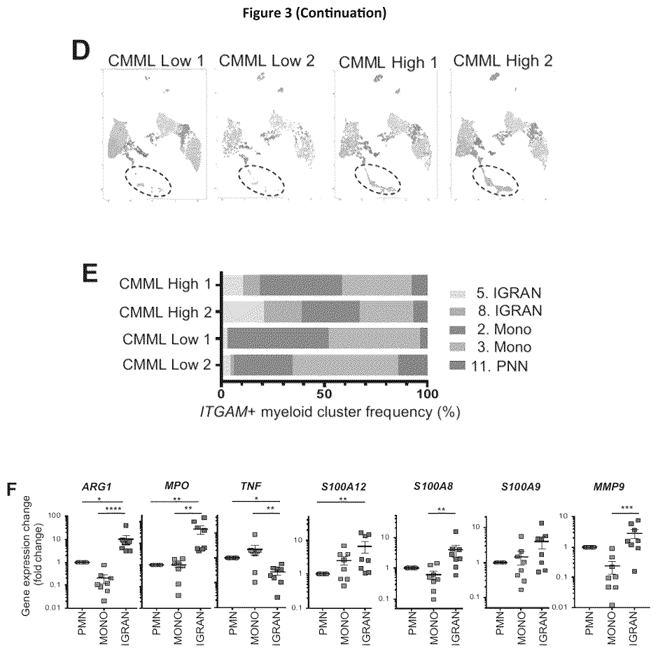

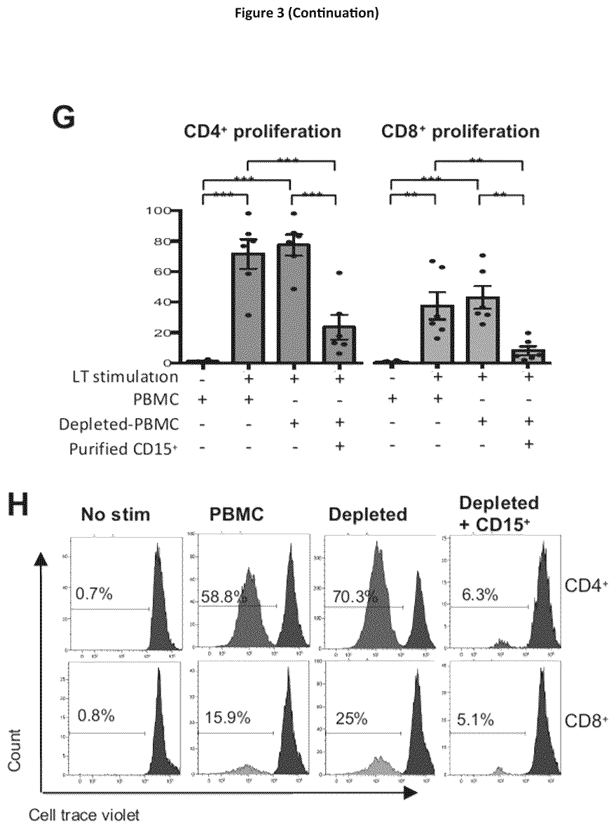

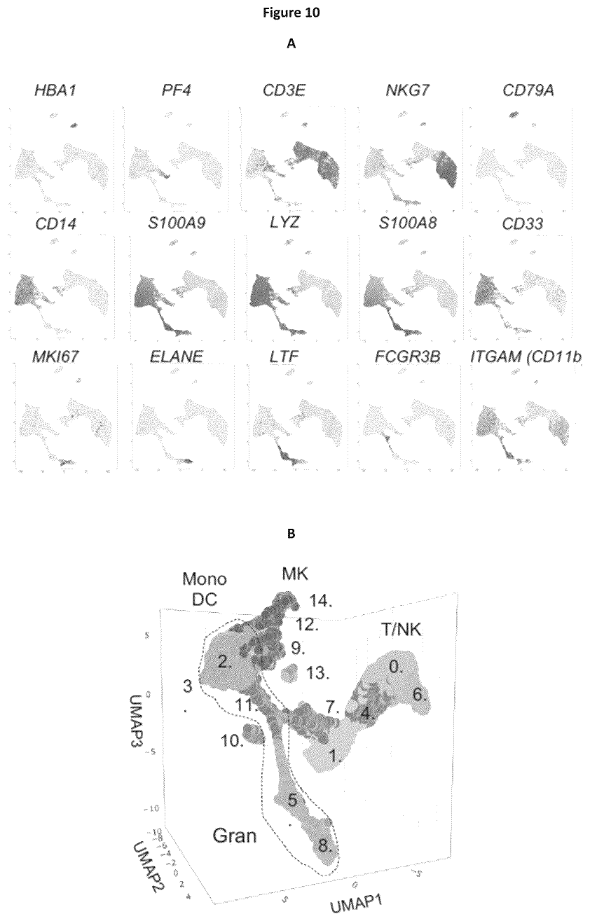

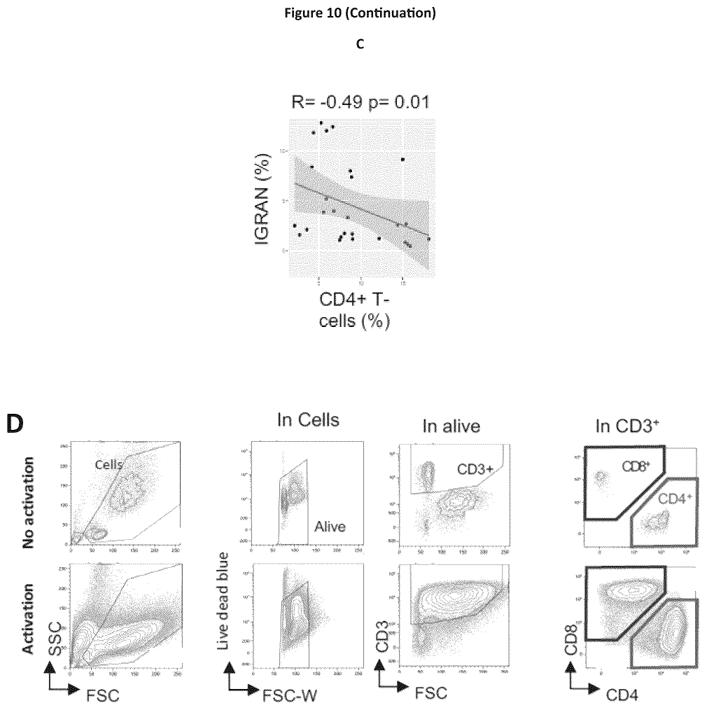

Having identified the poor prognostic significance of IGRAN excess, it was next sought to assess their functional impact. The phenotype of these cells (CD45^{low}w, CD33⁺, CD11b⁺, HLA-DR⁻, CD14⁻, CD15⁺, CD24⁺, CD66b⁺, and low density) suggested that they may be granulocytic myeloid-derived suppressor cells (G-MDSCs, also referred to as PMN-MDSCs) (FIG. 3A)(27). To explore this possibility, two patients were selected with an IGRAN-high (≥14%) and two patients with an IGRAN-low fraction and performed single-cell RNA sequencing analysis (scRNAseq) of PBMCs. The data were pooled and subjected to dimensionality reduction using the UMAP algorithm, and 15 clusters were identified (FIG. 3B). Based on the expression of marker genes, the cell types of clusters were identified, including T/NK cells (CD3E, NKG7), B cells (CD79A), erythroid cells (HBA1), megakaryocytes/platelets (PF4), monocytes (CD14, CD33), and granulocytes (FCGR3B, S100A9, S100A8, and LYZ) (FIG. 10 A-B). Granulocytes encompassed several clusters (11, 5, and 8), corresponding to sequential stages of neutrophil maturation. Cells in cluster 8 expressed genes encoding primary neutrophil granule proteins, such as ELANE, CEACAM, AZU1, DEFA1 and DEFA3, indicating promyelocytes and myelocytes (FIG. 3C, FIG. 10A). Cells in cluster 5 expressed genes encoding secondary and tertiary neutrophil granule proteins, such as MMP9, MMP8 and LTF, indicating metamyelocytes. Lastly, cells in cluster 11 expressed high levels of genes that characterize mature neutrophils, such as MME (CD10) and FCGR3B (CD16). Patients with an IGRAN-high fraction showed strong enrichment of cells in clusters 5 and 8 (FIGS. 3D and 3E), validating the initial description of this population as immature granulocytes.

The gene expression profile of human G-MDSC distinguishes them from mature neutrophils or monocytes (27). This profile was recapitulated in clusters 5 and 8 that exhibited high expression of cell cycle genes such as MKI67, as well as high expression of ARG1, MPO, S100A8, ANXA1, CYBB, and S100A12 genes and did not express the TNF gene (FIG. 3B). This gene signature was validated by RT-qPCR analysis in sorted CD16⁺ neutrophils, CD14⁺ monocytes and CD15⁺CD16⁻ IGRANs collected from 8 CMML patients. IGRANs expressed significantly higher levels of ARG1, S100A12 and MPO; similar levels of S100A8, S100A9 and MMP9; and lower levels of TNF compared to mature neutrophils or monocytes (FIG. 3F). These features are consistent with the hypothesis of G-MDSC (27,28).

The main characteristic of G-MDSCs is their ability to suppress immune cells. Consistent with the spectral flow cytometry analyses showing a significant inverse correlation between the IGRAN and CD4⁺ T-cell fractions (r=−0.49, p=0.01, FIG. 10C), conventional flow cytometry analysis showed an inverse correlation between the IGRAN and lymphoid cell fractions (obtained by WBC count). PBMCs collected from 6 untreated CMML patients were cultured for 4 days with anti-CD3 and anti-CD28 antibodies for T cell activation. The addition of IGRANs in the cultures inhibited CD4+ or CD8+ T-cell proliferation (FIG. 3G-H; FIG. 10 D. Taken together, these results demonstrate that IGRANs are G-MDSCs.

IGRANs are Clonal Cells with High Inflammatory Activity

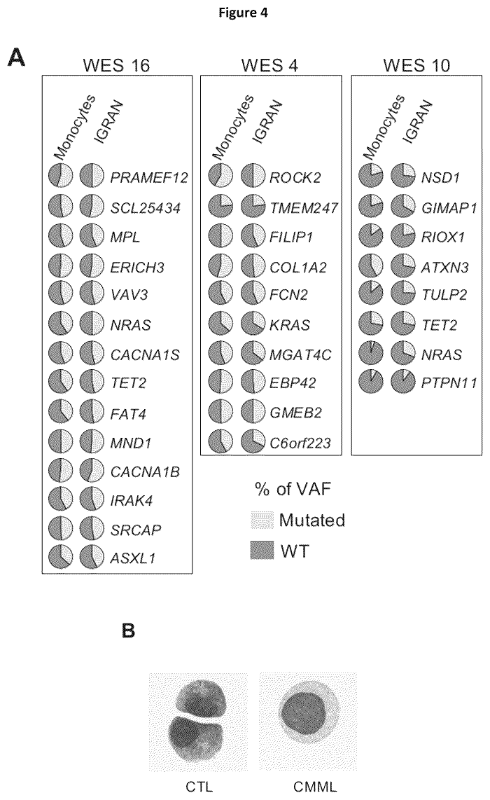

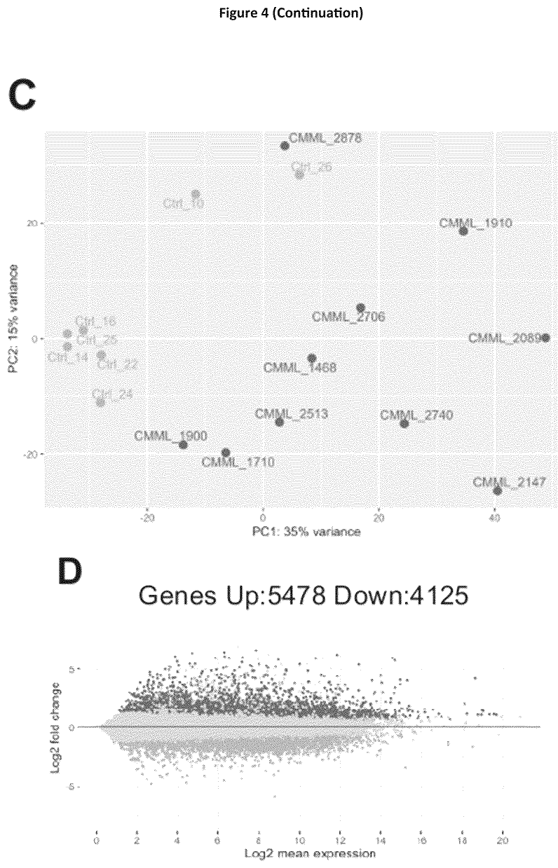

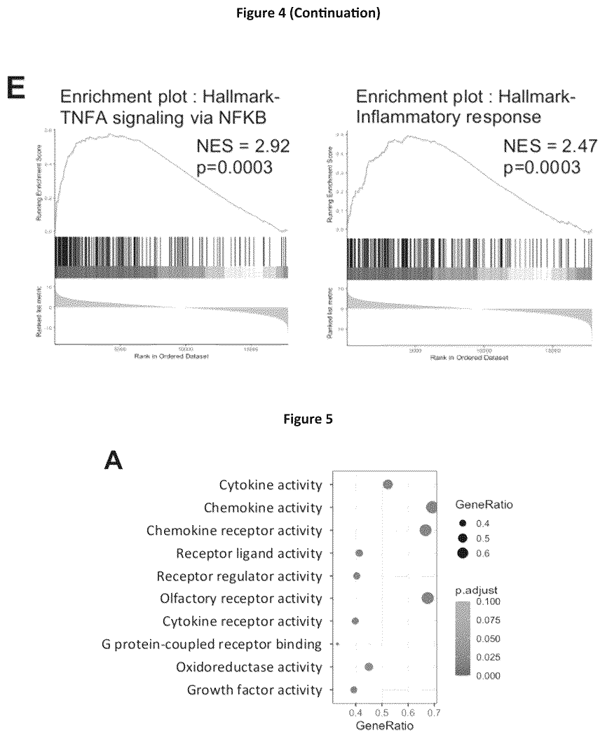

To further explore the link between IGRANs and the somatic genetic abnormalities that characterize CMML, whole exome sequencing of sorted IGRANs, monocytes and T cells was performed from 14 untreated CMML patients. For each patient analyzed, the somatic variants identified in IGRANs matched those detected in monocytes with similar variant allele frequencies (VAFs) (FIG. 4A), indicating that IGRAN accumulation is not related to subclonal coding region variants.

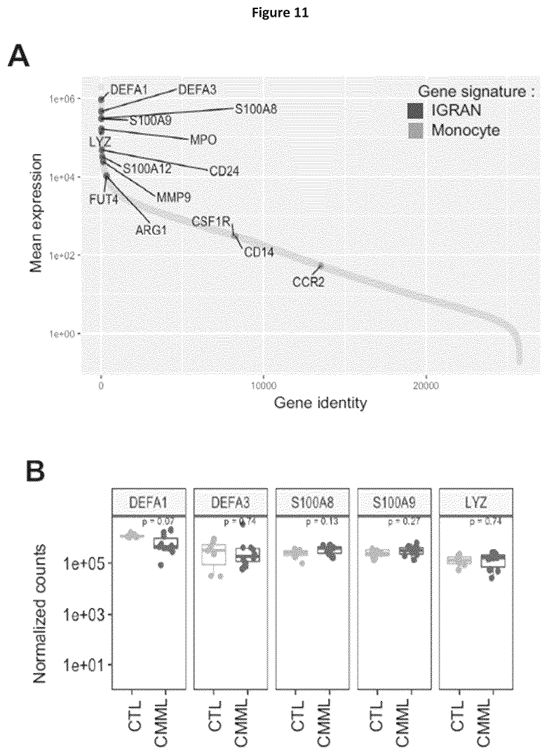

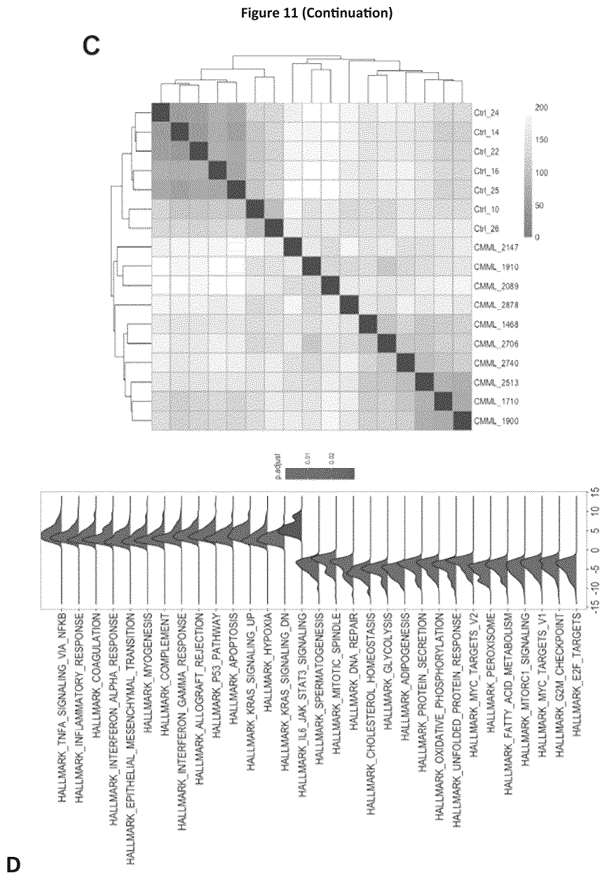

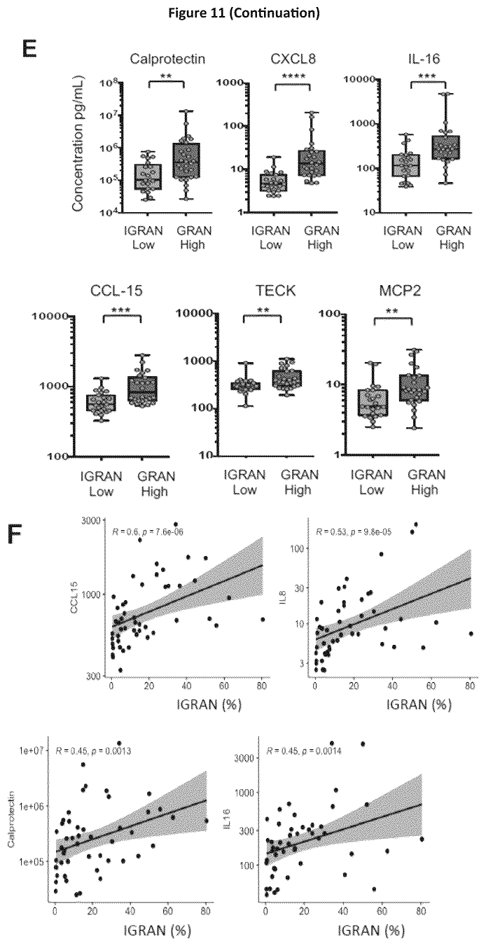

To characterize further CMML-associated clonal IGRANs, they were compared to sorted CD15⁺CD16⁻ cells collected from healthy donor by cytapheresis after mobilization. Cytological examination validated the immature morphology of collected cells, corresponding to promyelocytes or myelocytes, and revealed greater dysplasia in CMML IGRANs, which showed a loss of cytoplasmic granules and less condensed nuclear chromatin (FIG. 4B). Transcriptomic profiles were studied in CD15⁺CD16⁻ cells sorted from 7 healthy donors and 10 untreated CMML patients. Gene expression in the 17 pooled samples was ranked, and genes such as DEFA1, DEFA3, S100A9, MPO, and LYZ, were identified as the most highly expressed genes of the transcriptomic profile (FIG. 11 A), whether for CMML and control samples (FIG. 11B). Again these analyses validated the immature and granulocytic phenotype of the sorted cells.

Principal component analysis (PCA) showed that patient and control cells mostly clustered separately (FIG. 4C, FIG. 11 C). Based on an adjusted P value of <0.05 and a log 2-fold change >2, a total of 9,603 genes were found to be differentially expressed in CMML and control samples, of which 5,478 were upregulated (FIG. 4D). Pathway analysis using gene set enrichment analysis (GSEA) for hallmarks identified 33 pathways significantly enriched in CMML IGRANs. The pathways with the highest enrichment scores were TNFA_signaling_via_NFKB, inflammatory response, and interferon response, whereas various metabolic pathways were downregulated (FIG. 4E, FIG. 11D). These results indicate a proinflammatory status of clonal IGRANs in CMML patients.

### CXCL8 is the Main Cytokine Secreted by IGRAN

GSEA molecular function analysis of RNA sequencing data showed the significant enrichment of gene sets associated with receptor binding and cytokine and chemokine activity in CMML-associated IGRANs (FIG. 5A, B). Analysis of individual cytokine-encoding genes identified the CXCL8 gene (C—X—C motif chemokine ligand 8, also known as interleukin-8 or IL-8) as the most highly overexpressed (FIG. 5C, D). CXCL8 overexpression was confirmed by RT-qPCR analysis of two different sets of control and CMML cells (FIG. 5E). Then, the plasma levels of 44 cytokines and chemokines were measured in IGRAN-low (n=23) and IGRAN-high (n=26) CMML patients (FIG. 5F) and it was found that CXCL8 was the cytokine present at the highest level in the plasma of IGRAN-high CMML patients. CCL15 (C—C motif chemokine ligand 15), IL-16, the S100A8/S100A9 heterodimer (also known as calprotectin), MCP2 (monocyte chemoattractant protein 2, also known as CCL8), and TECK (CCL25) were also detected at significantly higher levels in the plasma of the IGRAN-high patient group compared to the IGRAN-low patient group (FIG. 11 E). Importantly, CXCL8, CCL15, calprotectin and IL16 plasma levels correlated with the IGRAN fraction detected by flow cytometry in CMML peripheral blood (FIG. 11F), IL-16, calprotectin and CXCL8 were also detected at high concentrations in the culture supernatant of sorted CMML-associated IGRANs (FIG. 5F) Together, these results identify CXCL8 as the most abundant cytokine secreted by IGRANs in CMML patients.

According to these results, IGRANs are part of a dialog between clonal mature cells and HSPCs. When the occurrence of a somatic mutation in a single HSC leads to clonal outgrowth, mature myeloid cells from the clone commonly demonstrate an inflammatory phenotype and promote multiple diseases (38-40), including myeloid malignancies (41). In the context of CMML, monocytes have been shown to secrete cytokine-like 1 (CYTL1), which reduces monocyte apoptosis through an autocrine or paracrine pathway involving MCL-1 and the MAPK pathway (42), while MIF, which is released in the context of TET2 truncation mutations, promotes the monocytic differentiation of HSPCs in a feed-forward loop (10). The capacity of IGRANs to produce increased amounts of CXCL8 that specifically prevents wildtype CD34⁺ cell expansion is reminiscent of a zebrafish model of clonal hematopoiesis in which myeloid cells derived from mutant HSPCs secrete inflammatory cytokines that repress the growth of wildtype HSCs but do not impact mutated HSCs (43). The inhibitory effect of CXCL8 on wildtype CD34⁺ cell growth provides a mechanistic explanation to the early clonal dominance that characterizes CMML clonal architecture (8).

### CXCL8 Receptor Inhibitors Restores the Growth of Wildtype CD34⁺ Cells

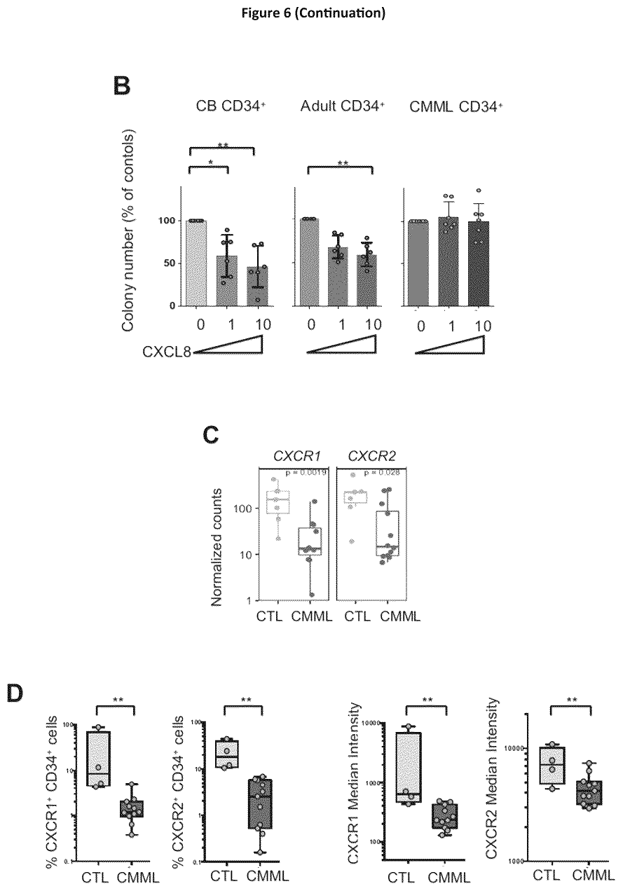

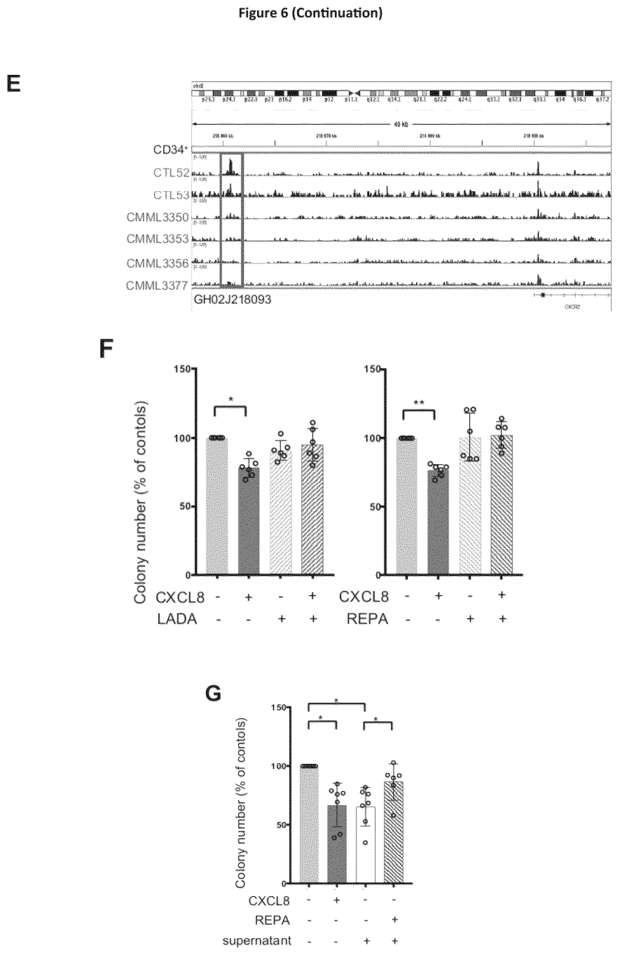

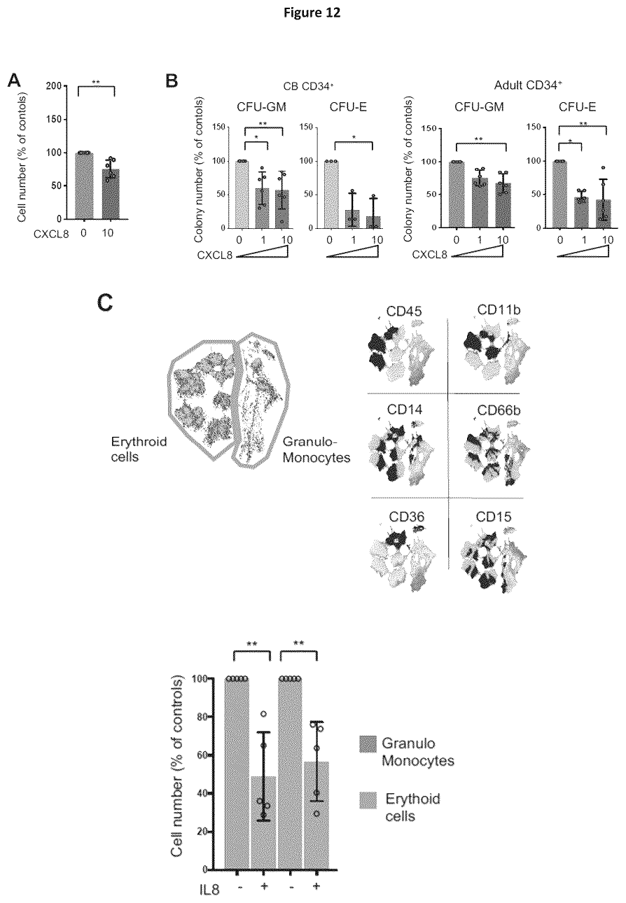

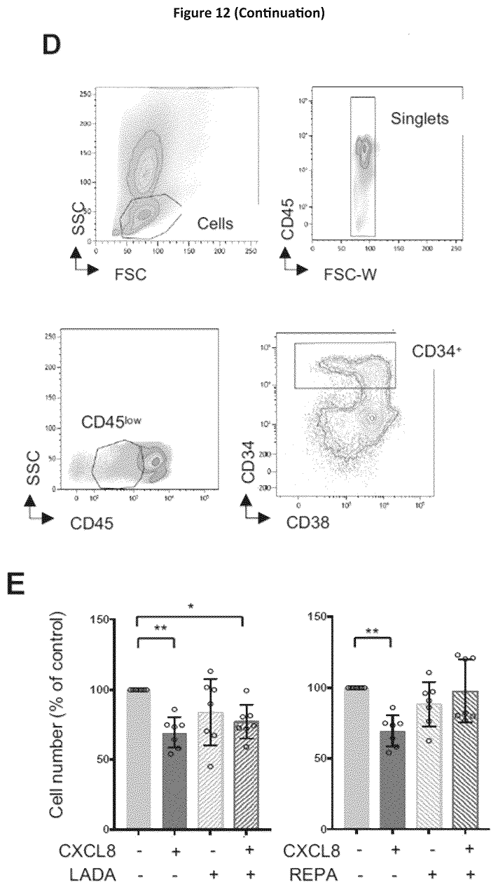

In CMML, the majority of hematopoietic stem and progenitor cells (HSPC) are clonal, mutated cells, with a low number of residual wildtype cells. As CXCL8 was the most abundant cytokine detected in the plasma of IGRAN-high patients, the impact of CXCL8 was evaluated on primary wildtype CD34⁺ cells collected from healthy donors (cord blood and adult bone marrow) and from primary CD34⁺ cells from CMML patient bone marrow. In liquid culture, a dose-dependent inhibition of cord blood CD34⁺ cell proliferation was observed in the presence of CXCL8 (FIG. 6A); a similar inhibitory effect was observed using adult healthy donor bone marrow CD34⁺ cells cultured with CXCL8 (FIG. 12A), In contrast, CXCL8 failed to inhibit CMML CD34⁺ cell proliferation. Consistent with these results, methylcellulose colony formation assays showed a decrease in colony number when healthy, cord blood or bone marrow CD34⁺ cells were seeded in the presence of CXCL8 (FIG. 6B), This decrease affected CFU-GM (granulocyte-monocyte colony-forming unit) and CFU-E (erythrocyte-colony-forming unit) number, as observed by microscopic visualization (FIG. 12B) and validated by spectral flow cytometry analysis of the colony phenotype (FIG. 12C), Again, in these assays, CMML CD34⁺ cell proliferation was not affected by the presence of CXCL8. Together, CXCL8 inhibits the growth and differentiation of wildtype myeloid CD34⁺ cells without impacting CMML CD34⁺ cells.

This lack of a response of CMML CD34⁺ cells to CXCL8 correlated with decreased expression of the CXCL8 receptors CXCR1 and CXCR2 at both the mRNA (Figure C) and protein levels (FIG. 6D, FIG. 12D). More precisely, the fraction of CD34⁺ cells expressing CXCR1 and CXCR2 at the cell membrane and the median fluorescence intensity of CXCR1 and CXCR2 were decreased in the CMML group compared to the healthy donor group. Searching for a mechanism at the origin of this receptor disappearance, ATAC-seq experiments were performed in CD34⁺ cells from controls and CMML patients. A peak corresponding to an open chromatin region was identified at the CXCR2 enhancer locus in control CD34⁺ cells but not in CMML CD34⁺ cells, suggesting that CXCR2 enhancer could be less accessible in the latter (FIG. 6E).

The lack of response of clonal HSPCs to CXCL8 was related to the downregulation of the CXCL8 receptors CXCR1 and CXCR2 at the surface of these cells. In some situations, mutant HSCs were shown to resist the chronic inflammation that otherwise triggers the exhaustion of nonmutated HSCs by switching from canonical to noncanonical NF-κB signaling (45). In the context of acute myeloid leukemia for example, IL-1 secreted by monocytes and myeloid blast cells promotes the growth and clonogenic potential of pathogenic CD34⁺ cells while suppressing colony formation by wildtype CD34⁺ cells (46). Another example is the ability of JAK2-mutated (47) and TET2-mutated (48) HSPCs to resist the suppressive effect of TNF on wildtype HSPCs. In CMML, the DNA methylation pattern of clonal cells plays an essential role in the disease phenotype (49). In the present study, the disappearance of a peak corresponding to an open chromatin region at the CXCR2 enhancer locus in CMML CD34⁺ cells may account for the decreased expression of the receptor.

In an attempt to counteract the negative effect of IGRAN-derived CXCL8 on wildtype CD34⁺ cell growth, the impact of pharmacologic inhibition of CXCL8 receptors, CXCR1 and CXCR2 was studied. Among the various small molecule CXCR1/2 antagonists that are being developed clinically, two were tested, namely ladarixin (4-[(2R)-1-oxo-1-(methanesulfonamide)])(29) and reparixin (R(−)-2-(4-isobutylphenyl)propionyl methanesulfonamide)(30). Consistent with previous results, the presence of CXCL8 in culture decreased colony forming capacity of wildtype CD34+ cells (FIG. 6F) as well as their proliferation in liquid culture (FIG. 12E). The addition of ladarixin or reparixin in combination with CXCL8 restored the proliferation and the colony forming capacity of wildtype CD34⁺ cells. Supernatant from IGRANs mimicked the impact of CXCL8 on healthy donor CD34⁺ colony formation, and reparixin blocked the inhibitory effects of this supernatant (e6G).

Together, CXCL8 secreted by IGRANs that accumulate in the peripheral blood of CMML patients inhibits the growth of wildtype CD34⁺ cells while sparing CMML CD34⁺ cells in which CXCL8 receptors are down-regulated, and pharmacological agents targeting CXCL8 receptors can protect wildtype CD34⁺ cells from CXCL8 inhibitory effect.

Those skilled in the art will understand that this invention may be embodied in many different forms and should not be construed as limited to the embodiments set forth in this application. Rather, these embodiments are provided so that this disclosure will fully convey the invention to those skilled in the art. Many modifications and other embodiments of the invention will come to mind in one skilled in the art to which this invention pertains having the benefit of the teachings presented herein.

The contents of all references, pending patent applications and published patents, cited throughout this application are hereby expressly incorporated by reference.

### BIBLIOGRAPHIC REFERENCES

1. Solary E, Itzykson R. How I treat chronic myelomonocytic leukemia. Blood. 2017; 130:126-36.
2. Khoury J D, Solary E, Abla O, Akkari Y, Alaggio R, Apperley J F, et al. The 5th edition of the World Health Organization Classification of Haematolymphoid Tumours: Myeloid and Histiocytic/Dendritic Neoplasms. Leukemia. Nature Publishing Group; 2022; 36:1703-19.
3. Merlevede J, Droin N, Qin T, Meldi K, Yoshida K, Morabito M, et al. Mutation allele burden remains unchanged in chronic myelomonocytic leukaemia responding to hypomethylating agents. Nat Commun. 2016; 7:10767.
4. Cervera N, Itzykson R, Coppin E, Prebet T, Murati A, Legall S, et al. Gene mutations differently impact the prognosis of the myelodysplastic and myeloproliferative classes of chronic myelomonocytic leukemia. Am i Hematol. 2014; 89:604-9.
5. Guru Murthy G S, Dhakal I, Mehta P. Incidence and survival outcomes of chronic myelomonocytic leukemia in the United States. Leuk Lymphoma. 2017; 58:1648-54.
6. Robin M, de Wreede L C, Padron E, Bakunina K, Fenaux P, Koster L, et al. Role of allogeneic transplantation in chronic myelomonocytic leukemia: an international collaborative analysis. Blood. 2022; 140:1408-18.
7. Itzykson R, Santini V, Thepot S, Ades L, Chaffaut C, Giagounidis A, et al. Decitabine Versus Hydroxyurea for Advanced Proliferative Chronic Myelomonocytic Leukemia: Results of a Randomized Phase III Trial Within the EMSCO Network. i Clin Oncol Off i Am Soc Clin Oncol. 2022; JC02200437.
8. Itzykson R, Kosmider O, Renneville A, Morabito M, Preudhomme C, Berthon C, et al. Clonal architecture of chronic myelomonocytic leukemias. Blood. 2013; 121:2186-98.
9. Padron E, Painter J S, Kunigal S, Mailloux A W, McGraw K, McDaniel J M, et al. GM-CSF-dependent pSTAT5 sensitivity is a feature with therapeutic potential in chronic myelomonocytic leukemia. Blood. 2013; 121:5068-77.
10. Pronier E, Imanci A, Selimoglu-Buet D, Badaoui B, Itzykson R, Roger T, et al. Macrophage migration inhibitory factor is overproduced through EGR1 in TETlow resting monocytes. Commun Biol. Nature Publishing Group; 2022; 5:1-15.
11. Lucas N, Duchmann M, Rameau P, Noël F, Michea P, Saada V, et al. Biology and prognostic impact of clonal plasmacytoid dendritic cells in chronic myelomonocytic leukemia. Leukemia. 2019; 1.
12. Niyongere S, Lucas N, Zhou J-M, Sansil S, Pomicter A D, Balasis M E, et al. Heterogeneous expression of cytokines accounts for clinical diversity and refines prognostication in CMML. Leukemia. 2019; 33:205-16.
13. Solary E, Itzykson R. Chronic Myelomonocytic Leukemia Gold Jubilee. Hemato. Multidisciplinary Digital Publishing Institute; 2021; 2:403-28.
14. Franzini A, Pomicter A D, Yan D, Khorashad J S, Tantravahi S K, Than H, et al. The transcriptome of CMML monocytes is highly inflammatory and reflects leukemia-specific and age-related alterations. Blood Adv. 2019; 3:2949-61.
15. Reynaud D, Pietras E, Barry-Holson K, Mir A, Binnewies M, Jeanne M, et al. IL-6 controls leukemic multipotent progenitor cell fate and contributes to chronic myelogenous leukemia development. Cancer Cell. 2011; 20:661-73.
16. Rai S, Grockowiak E, Hansen N, Luque Paz D, Stoll C B, Hao-Shen H, et al. Inhibition of interleukin-1β reduces myelofibrosis and osteosclerosis in mice with JAK2-V617F driven myeloproliferative neoplasm. Nat Commun. Nature Publishing Group; 2022; 13:5346.
17. Welner R S, Amabile G, Bararia D, Czibere A, Yang H, Zhang H, et al. Treatment of chronic myelogenous leukemia by blocking cytokine alterations found in normal stem and progenitor cells. Cancer Cell. 2015; 27:671-81.
18. Palmer L, Briggs C, McFadden S, Zini G, Burthem J, Rozenberg G, et al. ICSH recommendations for the standardization of nomenclature and grading of peripheral blood cell morphological features. Int J Lab Hematol. 2015; 37:287-303.
19. Patnaik M M, Padron E, LaBorde R R, Lasho T L, Finke C M, Hanson C A, et al. Mayo prognostic model for WHO-defined chronic myelomonocytic leukemia: ASXL1 and spliceosome component mutations and outcomes. Leukemia. 2013; 27:1504-10.
20. Becht E, McInnes L, Healy J, Dutertre C-A, Kwok I W H, Ng L G, et al. Dimensionality reduction for visualizing single-cell data using UMAP. Nat Biotechnol. 2018;
21. Selimoglu-Buet D, Wagner-Ballon O, Saada V, Bardet V, Itzykson R, Bencheikh L, et al. Characteristic repartition of monocyte subsets as a diagnostic signature of chronic myelomonocytic leukemia. Blood. 2015; 125:3618-26.
22. Selimoglu-Buet D, Badaoui B, Benayoun E, Toma A, Fenaux P, Quesnel B, et al. Accumulation of classical monocytes defines a subgroup of MDS that frequently evolves into CMML. Blood. 2017; 130:832-5.
23. Tarfi S, Badaoui B, Freynet N, Morabito M, Lafosse J, Toma A, et al. Disappearance of slan-positive non-classical monocytes for diagnosis of chronic myelomonocytic leukemia with an associated inflammatory state. Haematologica. 2020; 105:e147-52.
24. Itzykson R, Kosmider O, Renneville A, Gelsi-Boyer V, Meggendorfer M, Morabito M, et al. Prognostic score including gene mutations in chronic myelomonocytic leukemia. J Clin Oncol Off J Am Soc Clin Oncol. 2013; 31:2428-36.
25. Such E, Germing U, Malcovati L, Cervera J, Kuendgen A, Della Porta M G, et al. Development and validation of a prognostic scoring system for patients with chronic myelomonocytic leukemia. Blood. 2013; 121:3005-15.
26. Elena C, Galli A, Such E, Meggendorfer M, Germing U, Rizzo E, et al. Integrating clinical features and genetic lesions in the risk assessment of patients with chronic myelomonocytic leukemia. Blood. 2016; 128:1408-17.
27. Veglia F, Sanseviero E, Gabrilovich D I. Myeloid-derived suppressor cells in the era of increasing myeloid cell diversity. Nat Rev Immunol. 2021; 21:485-98.
28. Zhou J, Nefedova Y, Lei A, Gabrilovich D. Neutrophils and PMN-MDSCs: their biological role and interaction with stromal cells. Semin Immunol. 2018; 35:19-28.
29. Moriconi A, Cesta M C, Cervellera M N, Aramini A, Coniglio S, Colagioia S, et al. Design of noncompetitive interleukin-8 inhibitors acting on CXCR1 and CXCR2. J Med Chem. 2007; 50:3984-4002.
30. Bertini R, Allegretti M, Bizzarri C, Moriconi A, Locati M, Zampella G, et al. Noncompetitive allosteric inhibitors of the inflammatory chemokine receptors CXCR1 and CXCR2: prevention of reperfusion injury. Proc Natl Acad Sci USA. 2004; 101:11791-6.
31. Onida F, Barosi G, Leone G, Malcovati L, Morra E, Santini V, et al. Management recommendations for chronic myelomonocytic leukemia: consensus statements from the SIE, SIES, GITMO groups. Haematologica. 2013; 98:1344-52.
32. Wu Y, Yi M, Niu M, Mei Q, Wu K. Myeloid-derived suppressor cells: an emerging target for anticancer immunotherapy. Mol Cancer. 2022; 21:184.
33. Fang X, Xu S, Zhang Y, Xu J, Huang Z, Liu W, et al. Asxl1 C-terminal mutation perturbs neutrophil differentiation in zebrafish. Leukemia. 2021; 35:2299-310.
34. Braun T P, Estabrook J, Schonrock Z, Curtiss B M, Darmusey L, Macaraeg J, et al. Asxl1 deletion disrupts MYC and RNA polymerase II function in granulocyte progenitors. Leukemia. 2022;
35. Lee B H, Tothova Z, Levine R. Cell Rep. 2020; 33:108326.
40. Jaiswal S, Natarajan P, Silver A J, Gibson C J, Bick A G, Shvartz E, et al. Clonal hematopoiesis and risk for atherosclerotic cardiovascular disease. N Engl J Med. 2017; 377:111-21.
41. Jaiswal S, Fontanillas P, Flannick J, Manning A, Grauman P V, Mar B G, et al. Age-related clonal hematopoiesis associated with adverse outcomes. N Engl J Med. 2014; 371:2488-98.
42. Sevin M, Debeurme F, Laplane L, Badel S, Morabito M, Newman H L, et al. Cytokine-like protein 1-induced survival of monocytes suggests a combined strategy targeting MCL1 and MAPK in CMML. Blood. 2021; 137:3390-402.
43. Avagyan S, Henninger J E, Mannherz W P, Mistry M, Yoon J, Yang S, et al. Resistance to inflammation underlies enhanced fitness in clonal hematopoiesis. Science. 2021; 374:768-72.

L, Anderson K, Buza-Vidas N, Cullen D E, et al. FLT3 mutations confer enhanced proliferation and survival properties to multipotent progenitors in a murine model of chronic myelomonocytic leukemia. Cancer Cell. 2007; 12:367-80.
36. Smith M A, Choudhary G S, Pellagatti A, Choi K, Bolanos L C, Bhagat T D, et al. U2AF1 mutations induce oncogenic IRAK4 isoforms and activate innate immune pathways in myeloid malignancies. Nat Cell Biol. Nature Publishing Group; 2019; 21:640-50.
37. Weiss G, Ganz T, Goodnough L T. Anemia of inflammation. Blood. 2019; 133:40-50.
38. Fuster J J, MacLauchlan S, Zuriaga M A, Polackal M N, Ostriker A C, Chakraborty R, et al. Clonal hematopoiesis associated with TET2 deficiency accelerates atherosclerosis development in mice. Science. 2017; 355:842-7.
39. FusterJJ, Zuriaga M A, Zorita V, MacLauchlan S, Polackal M N, Viana-Huete V, et al. TET2-Loss-of-Function-Driven Clonal Hematopoiesis Exacerbates Experimental Insulin Resistance in Aging and Obesity
44. Padron E, Dezern A, Andrade-Campos M, Vaddi K, Scherle P, Zhang Q, et al. A Multi-Institution Phase I Trial of Ruxolitinib in Patients with Chronic Myelomonocytic Leukemia (CMML). Clin Cancer Res. 2016; 22:3746-54.
45. Muto T, Walker C S, Choi K, Hueneman K, Smith M A, Gul Z, et al. Adaptive response to inflammation contributes to sustained myelopoiesis and confers a competitive advantage in myelodysplastic syndrome HSCs. Nat Immunol. 2020; 21:535-45.
46. Carey A, Edwards D K, Eide C A, Newell L, Traer E, Medeiros B C, et al. Identification of Interleukin-1 by Functional Screening as a Key Mediator of Cellular Expansion and Disease Progression in Acute Myeloid Leukemia. Cell Rep. 2017; 18:3204-18.
47. Fleischman A G, Aichberger K J, Luty S B, Bumm T G, Petersen C L, Doratotaj S, et al. TNFα facilitates clonal expansion of JAK2V617F positive cells in myeloproliferative neoplasms. Blood. 2011; 118:6392-8.
48. Abegunde S O, Buckstein R, Wells R A, Rauh M J. An inflammatory environment containing TNFα favors Tet2-mutant clonal hematopoiesis. Exp Hematol. 2018; 59:60-5.
49. Selimoglu-Buet D, Rivière J, Ghamlouch H, Bencheikh L, Lacout C, Morabito M, et al. A miR-150/TET3 pathway regulates the generation of mouse and human non-classical monocyte subset. Nat Commun. 2018; 9:5455.
50. Arber D A, Orazi A, Hasserjian R, Thiele J, Borowitz M J, Le Beau M M, et al. The 2016 revision to the World Health Organization classification of myeloid neoplasms and acute leukemia. Blood. 2016; 127:2391-405.
51. Lausen B, Schumacher M. Maximally Selected Rank Statistics. Biometrics. [Wiley, International Biometric Society]; 1992; 48:73-85.
52. Hothorn T, Lausen B. On the exact distribution of maximally selected rank statistics. Comput Stat Data Anal. 2003; 43:121-37.
53. Bertini R. et al., Noncompetitive allosteric inhibitors of the inflammatory chemokine receptors CXCR1 and CXCR2: prevention of reperfusion injury. Proc Natl Acad Sci USA. 2004 Aug. 10; 101(32):11791-6. doi: 10.1073/pnas.0402090101. Epub 2004 Jul. 28.
54. Opfermann, P., Derhaschnig, U., Felli, A., Wenisch, J., Santer, D., Zuckermann, A., et al. (2015). A Pilot Study on Reparixin, a CXCR1/2 Antagonist, to Assess Safety and Efficacy in Attenuating Ischaemia-Reperfusion Injury and Inflammation after On-Pump Coronary Artery Bypass Graft Surgery. Clin. Exp. Immunol. 180 (1), 131-142. doi:10.1111/cei.12488
55. Liotti F. et al. Multiple anti-tumor effects of Reparixin on thyroid cancer Oncotarget. 2017 May 30; 8(22):35946-35961. doi: 10.18632/oncotarget.16412.
56. Schott A. F. et al., Phase Ib Pilot Study to Evaluate Reparixin in Combination with Weekly Paclitaxel in Patients with HER-2-Negative Metastatic Breast Cancer Clin Cancer Res. 2017 Sep. 15; 23(18):5358-5365.
57. Cao J. et al, Strategies to overcome myeloid cell induced immune suppression in the tumor microenvironment Front Oncol. 2023 April 11:13:1116016. doi: 10.3389/fonc.2023.1116016.
58. Mattos M. S. et al CXCR1 and CXCR2 Inhibition by Ladarixin Improves Neutrophil-Dependent Airway Inflammation in Mice Front. Irnmunol., 2 Oct. 2020 Sec. Cytokines and Soluble Mediators in Immunity Volume 11-2020.
59. Piro G. et al, CXCR1/2 dual-inhibitor ladarixin reduces tumour burden and promotes immunotherapy response in pancreatic cancer. Br J Cancer. 2023 January; 128(2):331-341. doi: 10.1038/s41416-022-02028-6

1. A method for treating chronic myelomonocytic leukemia (CMML), said method comprising administering to a patient in need thereof a pharmaceutical composition comprising an effective amount of an inhibitor of the CXCL8 receptor.

2. A method for maintaining or enhancing the proliferation of wildtype hematopoietic stem cells in patients suffering from CMML, said method comprising administering to a patient in need thereof a pharmaceutical composition comprising an effective amount of an inhibitor of the CXCL8 receptor.

3. The method of claim 1, wherein said CXCL8 receptor inhibitor inhibits the CXCL8 receptor CXCR1 and/or the CXCL8 receptor CXCR2.

4. The method of claim 1, wherein said CXCL8 receptor inhibitor is selected from anti-CXCL8 antibodies, AZD5069, SX-682, SX-576, SX-517, navarixin, ladarixin, meraxin, reparixin, reparixin L-lysine salt, DF2755A, a CXCL8 fragment comprising amino acids 3-74 and substitutions K11R/G31P (G31P), DF2162, SCH-479833, and combinations thereof.

5. The method of claim 1, wherein said CXCL8 receptor inhibitor is reparixin compound of formula:

6. The method of claim 1, wherein said CXCL8 receptor inhibitor blocks the CXCL8 receptor expressed by wildtype hematopoietic stem cells so as to enhance their proliferation in patients suffering from CMML.

7. The method of claim 1, wherein said pharmaceutical composition is administered orally at an effective dose of 400, 800, and 1200 mg three times a day.

8. The method of claim 1, further comprising administering a direct tumorigenic agent.

9. The method of claim 1, further comprising administering an agent that overcomes the suppressive potential of IGRANs.

10. A method for treating chronic myelomonocytic leukemia (CMML) in a subject in need thereof, said method comprising:
a) measuring the amount of immature granulocytes (IGRANs) in a sample of blood of said subject, and/or measuring the level of circulating CXCL8 in a sample of peripheral blood of said subject, and
b) administering a pharmaceutical composition containing an effective amount of an inhibitor of the CXCL8 receptor in subjects having high number of IGRANs and/or high level of circulating CXCL8 in their blood.

11. The method of claim 10, wherein step a) is performed by conventional flow cytometry or by single-cell RNA sequencing.

12. The method of claim 10, wherein step a) is performed by flow cytometry identification of CD45low, CD15+, CD16−, CD66b+ in the peripheral blood of said subject.

13. The method of claim 8, wherein the direct tumorigenic agent is a chemotherapeutic agent.
